(19) United States
(12) Patent Application Publication
(10) Pub. No.: US 2009/0023810 A1
(43) Pub. Date: Jan. 22, 2009

(54) METHODS OF TRANSLATION AND/OR INFLAMMATION BLOCKADE

(76) Inventors: Sung-Key JANG, Pohang-city (KR); Woo Jae Kim, Pohang-City (KR)

Correspondence Address: JHK LAW, P.O. BOX 1078, LA CANADA, CA, 91012-1078, US

(21) Appl. No.: 12/133,350

(22) Filed: Jun. 4, 2008

Related U.S. Application Data
(60) Provisional application No. 60/942,004, filed on Jun. 5, 2007.

(51) Int. Cl. A61K 31/20; C12Q 1/02; C07C 57/03; A61P 35/00; A61P 29/00

(52) U.S. Cl. 514/559; 435/29; 554/222

(57) ABSTRACT

The present invention relates to a method of translation or inflammatory response blockade by using a compound that binds to eIF4A, which is the 264^{th} amino acid residue, a method of developing an anti-inflammation, anti-cancer or anti-viral agent by screening a compound that binds to eIF4A.

### CROSS REFERENCE TO RELATED APPLICATION

This application claims priority to and the benefit of U.S. Provisional Application No. 60/942,004 filed on Jun. 5, 2007, which is hereby incorporated by reference for all purposes as if fully set forth herein.

### BACKGROUND OF THE INVENTION

(a) Field of the Invention

The present invention relates to a method of translation and/or inflammatory signaling blockade by using a compound that binds to eIF4A, a method of developing an anti-inflammation, anti-cancer or anti-viral agent by screening a compound that binds to eIF4A.

(b) Description of the Related Art

Inflammatory response can be considered a double-edged sword. It protects the body by triggering innate and acquired immunity under stress conditions such as tissue damage and infections, but chronic inflammatory responses can result in diseases such as cardiovascular disease, diabetes, arthritis, Alzheimer's disease, pulmonary disease, and autoimmune disease (Aggarwal et al., 2006). There are sophisticated mechanisms to maintain homeostatic inflammatory responses in animals and avoid adverse effects of inflammatory response (Lawrence et al., 2002).

Amines, complement, cyclic nucleotides, adhesion molecules, cytokines, chemokines, and steroid hormones are involved in regulation of inflammatory responses (Lawrence et al., 2002). Besides these factors, lipid mediators such as prostaglandins (PGs), leukotrienes, lipoxins, and resolvins play important roles in resolution of inflammation. Of various lipid mediators, prostaglandins are potent lipid molecules modulating immunity. The prostaglandins are a family of biologically active molecules with diverse actions depending on the prostaglandin type and cellular target. For instance, prostaglandin E2 (PGE2) provokes inflammatory responses; however, cyclopentone prostaglandins (cyPGs) such as 15-deoxy-delta 12,14-prostaglandin J2 (15d-PGJ2) and prostaglandin A1 (PGA1) inhibit inflammatory responses. cyPGs contain a cyclopentenone ring structure that forms a covalent bond with a cysteine residue in a target protein through a chemically reactive α,β-unsaturated carbonyl group. Various members of the cyPG family have anti-neoplastic, anti-inflammatory, and anti-viral activities (Straus and Glass, 2001). Recent research has indicated that cyPGs are endogenous anti-inflammatory mediators that promote the resolution of inflammation in vivo (Lawrence et al., 2002; Straus and Glass, 2001). In general, the production of pro-inflammatory prostaglandins such as PGE2 triggers and/or maintains inflammatory responses, and then follows the production of anti-inflammatory prostaglandins to prevent adverse effects of inflammatory responses.

15d-PGJ2 is produced in a variety of cells, including mast cells, T cells, platelets, and alveolar macrophages. Several activities of 15d-PGJ2 have been suggested. 15d-PGJ2 is an agonist of peroxisome proliferator-activated receptor-gamma (PPARγ), which is a transcriptional modulator that represses transcription of pro-inflammatory mRNAs, thereby resulting in resolution of inflammatory responses. Moreover, 15d-PGJ2 blocks pro-inflammatory NF-κB signaling cascades independently of PPARγ through direct interactions with signaling molecules (Straus et al., 2000). Other physiological activities of 15d-PGJ2, such as cytoprotection and inhibition of cell proliferation, have also been reported (Pereira et al., 2006). However, the molecular mechanisms involved in these activities remain obscure.

Translation initiation is a complex process that begins with interaction of the cap-binding protein complex eukaryotic initiation factor 4F (eIF4F) with the mRNA 5′-end cap structure. eIF4F comprises three subunits: eIF4E, a cap-binding protein; eIF4A, an RNA helicase; and eIF4G, a scaffolding protein. eIF4G bridges eIF4F with the 40S ribosomal subunit through an interaction with eIF3 that is associated with the 40S ribosomal subunit. The 40S ribosomal subunit with the associated initiation factors is thought to migrate along the 5′-nontranslated region (NTR) until it encounters the initiation codon AUG. The 40S ribosomal subunit stalls at the initiation codon and the 60S ribosomal subunit joins to form the 80S ribosomal complex. Following assembly of the 80S ribosome at the mRNA initiation codon, elongation of the polypeptide chain commences (Holcik and Sonenberg, 2005).

Translation initiation of most mRNAs is repressed when a cell is under stress conditions such as heat and oxidation. Blockade of translation by stress signals results in formation of stress granules (SGs) in the cytoplasm. SGs contain most of the components of the 48S translational pre-initiation complex (the small, but not the large, ribosomal subunits, namely eIF4A, eIF3, eIF4E, eIF4G, eIF2 and eIF2B), other RNA-binding proteins such as T-cell-restricted intracellular antigens-1 (TIA-1), T-cell-restricted intracellular antigen-related protein (TIAR), and mRNAs. Unlike other RNA granules, SGs are not observed in cells growing under favorable conditions but are rapidly induced in response to environmental stresses (Anderson and Kedersha, 2006). Transient inhibition of protein synthesis, which induces SG formation, is an important protective mechanism used in cells during various stress conditions such as inflammation (Ma and Hendershot, 2003).

Recently, a new role of SGs has been uncovered. The inventors showed that a signaling molecule TRAF2, which has a key role in tumor-necrosis factor α (TNF-α; a pro-inflammatory cytokine) signal transduction, is sequestered into the SGs induced by heat treatment through an interaction with the translational factor eIF4GI (Kim et al., 2005). Owing to SG formation, not only translation but also TNF-α signal transduction processes are blocked under heat-stress conditions. This phenomenon represents a novel relationship between translation and inflammatory signaling (Kim et al., 2005; McDunn and Cobb, 2005).

Here, the inventors present data on another crosstalk between translation and TNF-α signaling. The cyPGs 15d-PGJ2 and PGA1, which have anti-inflammatory activities, induce SG formation. However, PGE2, which has pro-inflammatory activity, does not induce SG formation. The SG formation was triggered by blockade of translational initiation by modification of the translational initiation factor eIF4A. Translational inhibition by 15d-PGJ2 is most likely related to the anti-cell-proliferation activity of 15d-PGJ2. Moreover, TRAF2 was sequestered to the SGs induced by cyPGs in a similar manner as it is to the SGs induced by heat treatment. This indicates that the anti-inflammatory activity of cyPGs is attributed in part to inhibition of translation and SG formation resulting in TRAF2 sequestration.

### SUMMARY OF THE INVENTION

An embodiment of the present invention provides a method of translation inhibition by administering a compound capable of binding to cysteine 264 of eIF4A to a subject in need of decrease of inflammatory response, to inactivate eIF4A.

Another embodiment of the present invention provides a method of anti-inflammation or anti-cancer by administering a compound capable of binding to cysteine 264 of eIF4A to a subject suffering with inflammation or cancer.

Still another embodiment of the present invention provides developing an anticancer or anti-inflammatory drug that targets cysteine 264 of eIF4A.

### BRIEF DESCRIPTION OF THE DRAWINGS

FIGS. 1A-1E show that cyPGs induce SG formation.

1A shows images of immunocytochemical analyses of HeLa cells that were mock-treated (Mock) or treated with PGA1, 15d-PGJ2, PGE2, SA, arachidonic acid (AA), ciglitazone (Ci), troglitazone (Tro), and rosiglitazone (Rosi) (at indicated concentrations) for 30 minutes, wherein the immunocytochemical analyses were performed using TIA-1 antibody.

1B shows images of immunocytochemical analyses of HeLa cells that were pretreated for 1 hour with 10 μg/ml of emetine and then treated with 15d-PGJ2 (50 μM) for 1 hour.

1C shows images of immunocytochemical analyses of HeLa cells that were treated with 15d-PGJ2 (50 μM) for 1 hour, wherein the immunocytochemical analyses were performed with the indicated antibodies: HuR/TIAR (a), HuR/eIF4A1 (b), PABP/TIA-1 (c), eIF4GI/rps6 (d), eIF4GI/L28 (e), and hsp27/eIF3b (f) (nuclei are shown in blue by Hoechst staining and arrows indicate SGs).

1D shows images of immunocytochemical analyses of HeLa cells that were treated with 15d-PGJ2 (10 μM) for the times indicated, wherein the immunocytochemistry was performed with an eIF3b (green) and HuR (red) antibodies (arrows indicate SGs).

1E shows images of immunocytochemical analyses of HeLa cells that were treated with heat at 44° C. (b, f, and j), 50 μM of PGE2 (c, g, and k), or 15d-PGJ2 (d, h, and 1) for 1 hour, wherein the immunocytochemistry was performed using eIF3b and TRAF2 antibodies (arrows indicate SGs).

FIGS. 2A-2E show that SG formation by 15d-PGJ2 is independent of eIF2α phosphorylation and TIA-1 aggregation.

2A shows the phosphorylated eIF2α levels that were monitored by Western-blot analyses using HeLa cell extracts (40 μg) treated with 15d-PGJ2 (lanes 2-4), PGA1 (5), PGE2 (6), Rosi (7), or SA (8) at the indicated concentrations for 30 minutes or with heat at 44° C. for 30 minutes.

2B shows images of immunocytochemical analyses of HeLa cells grown on cover slips and pretreated with 1 mM of 2-AP or with vehicle for 6 hours, and then treated with 50 μM of 15d-PGJ2 for 30 minutes, wherein fixed cells were analyzed by immunocytochemistry with an eIF3b antibody.

2C shows images of immunocytochemical assays of wild-type and eIF2α A/A mutant MEF cells that were treated with 400 μM of SA for 30 minutes or 50 μM of 15d-dPJ2 for 1 hour, wherein the immunocytochemical assays were performed with a TIA-1 antibody.

2D shows images of immunocytochemical assays of wild-type, TIA-1 KO, and TIAR KO MEF cells that were mock-treated (upper panel) or treated with 15d-PGJ2 (lower panel), wherein the immunocytochemical analyses were performed with an eIF3b antibody.

2E shows images of immunocytochemical assays of HeLa cells transfected with a plasmid encoding FLAG tagged-eIF2α S51A, wherein After 48 hours of incubation, cells were mock-treated (left), treated with 400 μM of SA (middle) or with 50 μM of 15d-PGJ2 (right) for 30 minutes, and the loci of eIF4GI and eIF2α S51A were visualized by an immunocytochemical method using eIF4GI and FLAG antibodies, respectively.

FIGS. 3A-3D show that SG formation by 15d-PGJ2 is independent of PPARγ.

3A shows images of immunocytochemical analyses of HeLa cells that were grown on cover slips and transfected with a siRNA against PPARγ (b and e) or a plasmid pTR100-PPARγ expressing high levels of PPARγ (c and f), wherein after transfection, cells were treated with 50 μM of 15d-PGJ2 for 1 hour and the immunocytochemical analyses were performed with eIF3b and PPARγ antibodies, shown in green and red, respectively.

3B shows the amounts of PPARγ in cells transfected with control siRNA (lane 1), siRNA against PPARγ (lane 2) and pTR100-PPARγ (lane 3), which were analyzed by Western-blot assays using a PPARγ antibody, wherein lysates were normalized by an actin blot.

3C shows images of immunocytochemical analyses of HeLa cells that were pretreated with 1 μM of GW9662, an irreversible PPARγ antagonist, for 24 hours and then treated with SA (400 μM), PGE2 (50 μM), 15d-PGJ2 (50 μM), or PGA1 (50 μM) for 1 hour, wherein the immunocytochemical analyses were performed with eIF3b and HuR antibodies, shown in green and red, respectively, and the nuclei are shown in blue by Hoechst staining.

3D shows relative luciferase activities in the cell extracts normalized to a mock-treated control extract.

FIGS. 4A-4F show that 15d-PGJ2 and PGA1 inhibit translation in vivo.

4A shows images of immunocytochemical analyses of HeLa cells that were grown on 60-mm dishes up to about 70-80% confluence, wherein cells were mock-treated (1) or treated with PGA1 (2, 3, and 4), 15d-PGJ2 (5 and 6 and 7), or PGE2 (8, 9, and 10) at indicated the concentrations for 30 minutes then in vivo labeling of newly synthesized proteins was performed as described in Materials and Methods. 4200 CPM was obtained from the TCA-precipitated control sample (lane 1), and phosphorylated eIF2α levels were monitored by Western-blot analyses (bottom panel).

4B shows images of immunocytochemical analyses of Cells that were mock-treated (1), treated with SA (400 μM) (2 and 3), with PGA1 (90 μM) (4-6), with 15d-PGJ2 (90 μM) (7-9), and with PGE2 (90 μM) (10-12) as indicated times. Newly synthesized proteins were measured as panel (A), wherein 4500 CPM was obtained from the TCA-precipitated control sample (lane 1) and phosphorylated eIF2α levels were monitored by Western-blot analyses (bottom panel).

4C shows images of immunocytochemical analyses of HeLa cells that were mock-treated or treated with SA (400 μM) for 30 minutes, 15d-PGJ2 (50 μM) for 1 hour, or PGE2 (50 μM) for 1 hour, wherein sucrose gradient experiment was performed as described in Materials and Methods, and the lines show observance at 254 nm.

4D-4F shows effects of LPS on translation in RAW264.7 macrophage cells.

4D shows images of immunocytochemical analyses of RAW264.7 cells that were incubated with LPS for 24 hours at the indicated concentrations, wherein after the LPS treatment, mRNAs (1 μg) containing Renilla luciferase translated in a cap-dependent manner and mRNAs (1 μg) containing firefly luciferase under the control of cricket paralysis virus (CrPV) IRES were co-transfected into the cells.

4E shows images of immunocytochemical analyses of RAW264.7 cells that were incubated with LPS (10 μg/ml) for the times indicated, wherein transfection of mRNAs and analyses of luciferase activities were performed as described in panel (D).

4F shows images of immunocytochemical analyses of RAW 264.7 cells that were pretreated (white columns) or not pretreated (grey columns) with indomethacin (1 μM) for 30 min before being treated with LPS (10 μg/ml), wherein transfection of mRNAs and analyses of luciferase activities were performed as described in panel (D).

FIGS. 5A-5C show that PGA1 and 15d-PGJ2 inhibit translation in vitro.

5A shows the results of [³⁵S]-labeling experiment to quantify β-gal mRNA (40 nM) that was translated in HeLa lysates for 1 h in the presence of PGA1 (2), 15d-PGJ2 (3) and PGE2 (4) at indicated concentrations, wherein the [³⁵S]-labeling experiment was performed as described by Pestova et al. (Pestova et al., 1998).

5B shows relative luciferase activities and phosphorylated eIF2α levels.

5C shows relative in the translation mixtures containing various compounds were normalized to those in mock-treated extracts with the corresponding mRNAs, and are shown as columns (mean values).

FIGS. 6A-6E shows that eIF4A is the target of 15d-PGJ2.

6A shows images of Immunocytochemical analyses of HeLa cells that were grown on cover slips and then treated with biotinylated 15d-PGJ2 (50 μM; a, b, d, e, g, and h) or biotinylated PGE2 (50 μM; c, f, and i) for 1 hour, wherein the immunocytochemical analyses were performed with primary antibodies against eIF3b (a, c, d, f, g, and i) and L28 antibodies (b, e, and h). Biotinylated chemicals were visualized with FITC-conjugated streptavidin, and arrows indicate SGs induced by biotinylated 15d-PGJ2.

6B shows luciferase activities in the translation mixtures containing 15d-PGJ2 that were normalized to those in the corresponding translation mixtures without 15d-PGJ2, and are shown as columns (mean values).

6C shows the results of western-blot analyses with antibodies against eIF4GI, eIF4AI, eIF4E, poly(A)-binding protein (PABP), and eIF3c on resin-bound proteins.

6D shows the results of Comassie blue staining on resin-bound proteins.

6E shows the results of immunoprecipitation on 293T cells that were transfected with the wild-type (WT, lane 1) or mutant (lanes 2-4) FLAG-eIF4A1s (provided by Dr. Yongjun Dang and Dr. Jian Liu, Johns Hopkins), wherein the immunoprecipitation was performed as described in Materials and Methods.

FIGS. 7A-7F show that 15d-PGJ2 blocks the interaction between eIF4G and eIF4A.

7A shows the results of western-blot analysis performed with anti-FLAG, anti-HA, and anti-eIF4GI antibodies on 293T cells that were co-transfected with HA-eIF4B and FLAG-eIF4 μl (provided by Dr. Yongjun Dang and Dr. Jian Liu, Johns Hopkins), wherein the cells were lysed then treated with 50 μM of PGE2 or 15d-PGJ2 at 30° C. for 1 hour, and immunoprecipitation was performed with an anti-FLAG antibody.

7B shows the results of western-blot analysis performed with antibodies against eIF4GI, eIF3b, PABP, eIF4AI, actin, or eIF4E on resin-bound proteins.

7C shows the results of silver staining (upper panel) or western-blot analysis with an anti-FLAG antibody (lower panel) on RNA-bound proteins.

7D shows luciferase activities with (2, 4, and 6) 15d-PGJ2 treatment compared with those without (1, 3, and 5) 15d-PGJ2 treatment in the presence of additional eIF4A at particular concentrations and are shown as columns (mean values).

7E shows images of immunocytochemical analyses of HeLa cells were grown on cover slips and transfected with a FLAG vector, plasmid FLAG-eIF4Awt expressing the wild type eIF4A tagged with FLAG, or plasmid FLAG-eIF4A^{C264S} expressing a C264S mutant eIF4A tagged with FLAG, wherein after 48 hours of incubation, cells were treated with the chemicals at the concentrations indicated for 30 minutes, and the immunocytochemical analyses were performed with eIF4GI and FLAG antibodies.

7F shows a hypothetical model of anti-inflammatory activities of 15d-PGJ2.

FIGS. 8A to 8D show that 15d-PGJ2 is a specific SG inducer among anti-inflammatory compounds.

8A shows immunocytochemical analyses of HeLa treated with lovastatin, lipoxin, lipoxin B4 (LXB4), epi-lipoxin A4 (epi-LxA4), TGF-β (100 ng/ml), or interleukin-10.

8B shows immunocytochemical analyses of HeLa grown on cover slips were treated with CAY10410 alone (lower panels) or together with 15d-PGJ2 (upper panels) for 30 minutes (left panels) or 60 minutes (right panels).

8C shows relative luciferase activities of Mock-, 15d-PGJ2-, or CAY10410-treated Hela lysates or RRL (rabbit reticulocyte lysate).

8D shows the chemical structures of 15d-PGJ2 and CAY10410

FIGS. 9A to 9C shows that 15d-PGJ2 induces SG formation in various cell lines.

9A shows immunocytochemical analyses of SH-SY5Y cells mock-treated or treated with 15d-PGJ2.

9B shows immunocytochemical analyses of RAW264.7 macrophage cells mock-treated or treated with 15d-PGJ2.

9C shows immunocytochemical analyses of HEK 293T cells mock-treated or treated with 15d-PGJ2.

FIGS. 10A and 10B shows that TRAF2, but not RIP and IKKα/β, is sequestered into SGs.

10A shows immunocytochemical analyses of HeLa cells mock-treated (left panels) or treated with 15d-PGJ2 (right panels).

10B shows immunoprecipitation results using anti-FLAG antibody on 293T cells co-transfected with plasmids expressing FLAG-tagged RIP and HA-tagged TRAF2.

FIG. 11 is a modified graph showing 15d-PGJ2 levels secreted from activated RAW264.7 macrophages.

FIGS. 12A and 12B shows effects of 15d-PGJ2 on HCV IRES-dependent translation and ribosome binding of mRNA.

12A shows HCV IRES-dependent translation depending on treating 15d-PGJ2 or eIF4A.

12B shows the results of ribosomal pull-down experiments.

### DETAILED DESCRIPTION OF THE PREFERRED EMBODIMENTS

A more complete appreciation of the invention, and many of the attendant advantages thereof, will be readily apparent as the same becomes better understood by reference to the following detailed description.

The signaling lipid molecule 15-deoxy-delta 12,14-prostaglandin J2 (15d-PGJ2) has multiple cellular functions including anti-inflammatory and anti-neoplastic activities. Here, the present inventors report that 15d-PGJ2 blocks translation through inactivation of translational initiation factor eIF4A. Binding of 15d-PGJ2 to eIF4A blocks the interaction between eIF4A and eIF4G that is essential for translation of many mRNAs. Cysteine 264 in eIF4A is the target site of 15d-PGJ2. The anti-neoplastic activity of 15d-PGJ2 is likely attributed to inhibition of translation. Moreover, inhibition of translation by 15d-PGJ2 results in stress granule (SG) formation, into which TRAF2 is sequestered. The sequestration of TRAF2 contributes to the anti-inflammatory activity of 15d-PGJ2. These findings reveal a novel crosstalk between translation and inflammatory response, and offer new approaches to develop anti-cancer and anti-inflammatory drugs that target translation factors including eIF4A.

An embodiment of the present invention provides a method of translation inhibition by treating a compound capable of blocking translational initiation factor eIF4A and eIF4G interaction, or a compound capable of binding to cysteine 264 of translational initiation factor eIF4A, to inactivate eIF4A. The eIF4A is a subunit of eIF4F, and may have the amino acid sequence of SEQ ID NO: 1 (accession no. NP_{—}001407)

As used herein, the term ‘cysteine 264 of eIF4A’ means cysteine that is positioned at 264th amino acid position of eIF4A.

The subject may be any mammal including the human beings. The compound may have a cyclopentenone ring that is capable of specifically recognizing cysteine 264 of eIF4A, and preferably, the compound may be 15-deoxy-delta 12,14-prostaglandin J2 (15d-PGJ2).

One of the characteristics of the present invention is to reveal the relationship between translation and inflammatory response or cancer. Therefore, another embodiment of the present invention provides a method of anti-inflammation and/or anti-cancer by administering a compound capable of binding to cysteine 264 of eIF4A to a subject suffering with inflammation or cancer. In the present invention, the anti-inflammation and/or anti-cancer effects can be achieved by inhibiting translation by blocking cysteine 264 of eIF4A, resulting stress granule formation in, into which TRAF2 (TNF receptor-associated factor 2) is sequestered. The stress granule formation is known to participate in the anti-inflammation and/or anti-cancer effects. The subject may be any mammal including the human beings The compound may have a cyclopentenone ring that is capable of specifically recognizing cysteine 264 of eIF4A, and preferably, the compound may be 15-deoxy-delta 12,14-prostaglandin J2 (15d-PGJ2).

One of the characteristics of the present invention is to reveal the relationship between translation and inflammatory response and/or cancer, and to provide eIF4A as a novel target to develop anticancer and/or anti-inflammatory drugs. Therefore, another embodiment of the present invention provides a method of developing anticancer and/or anti-inflammatory drugs that targets eIF4A. More specifically, the method may include the steps of:

contacting a candidate compound to an animal or plant cell,

measuring inhibition of translation initiated by translational initiation factor eIF4A in vivo or in vitro, and

determining the compound as an anti-cancer or anti-inflammatory drug when the compound causes the inhibition of translation.

The measuring step may be performed by any conventional in vivo and in vitro method known to the relevant technical field.

The inhibition of translation may be measured by observing stress granule formation in the cell. Alternatively, the inhibition of translation is measured by monitoring the presence of blockade of the interaction between eIF4A and eIF4G. In addition, the interaction between the compound and cysteine 264 of eIF4A may be monitored by determining stress granule formation. The interaction between the compound and cysteine 264 of eIF4A may also be monitored by in vivo labeling method or in vitro translation system. For example, the interaction between the compound and cysteine 264 of eIF4A may be monitored by comparing the translation level in the cell containing cysteine 264 of eIF4A to that of the cell where cysteine 264 of eIF4A is modified with other amino acid, such as serine. The cell may be obtained from any mammal including the human beings.

Recently, progress has been made in determining the interplay between translational processes and pro-inflammatory signaling (Kim et al., 2005; McDunn and Cobb, 2005). Here, the present inventors report that the anti-inflammatory signaling molecule 15d-PGJ2, which is known to block pro-inflammatory signaling, inhibits translation in vivo (FIG. 4) and in vitro (FIG. 5). Several lines of evidence suggest that, for translational inhibition, the main target of 15d-PGJ2 is the translational initiation factor eIF4A. First, 15d-PGJ2 directly binds to eIF4A1, as shown by pull-down experiments with biotinylated 15d-PGJ2, HeLa cell extracts (FIG. 6C) and purified eIF4A (FIG. 6D). Second, eIF4A has previously been identified as a cellular target of 15d-PGJ2 using a proteomic approach; however, the physiological importance of the eIF4A-15d-PGJ2 interaction was not reported (Aldini et al., 2007). Third, the translation inhibitory effect of 15d-PGJ2 was relieved by the addition of purified eIF4A1 (FIG. 7D). The amount of purified eIF4A1 required for complete restoration of translation was about 0.5 μM to final that is about 1/100 amount of 15d-PGJ2 by molarity in the reaction mixture. This suggests that restoration of translation is not due to nonspecific inactivation of 15d-PGJ2 by the newly added eIF4A1 in the translation reaction mixture, but is likely to be due to replacement of inactive 15d-PGJ2-conjugated eIF4A1 proteins with functional eIF4A1 proteins. Fourth, SG formation in HeLa cells by 15d-PGJ2, which reflects translational inhibition, was hampered by overproduction of eIF4A and its derivative (C264S mutant) (FIG. 7E). The C264S mutant, which lacks the binding site of 15d-PGJ2, showed stronger resistance to SG formation by 15d-PGJ2 than the wild type eIF4A. This suggests that SG formation by 15d-PGJ2 is induced by the binding of 15d-PGJ2 to eIF4A.

While investigating anti-proliferating agents, Low and co-workers found that a natural marine compound named pateamine A (PatA) could block translation by inactivating eIF4A (Low et al., 2005). Interestingly, like 15d-PGJ2, this compound also induces formation of SGs independently of eIF2F phosphorylation (Dang et al., 2006) and impairs ribosome binding to mRNA (FIGS. 12A and 12B) (Bordeleau et al., 2006b; Mazroui et al., 2006). The authors suggested that PatA inhibits translation by blocking the eIF4G-eIF4A interaction (Low et al., 2005). However, Pelletier and colleagues have suggested that RNA-mediated sequestration of eIF4A is the translational inhibitory mechanism of PatA (Bordeleau et al., 2006a). It is possible that sequestration of eIF4A into RNAs may also partly contribute to translational inhibition by 15d-PGJ2 because the RNA-binding activity of purified eIF4A was increased in the presence of 15d-PGJ2 (FIG. 7C). However, it should be noted that the eIF4A and eIF4G interaction was also blocked in the presence of RNase (FIG. 7A), indicating that 15d-PGJ2 actively blocks this protein-protein interaction.

Investigations into the role of the tumor suppressor protein named programmed cell death 4 (Pdcd4), which blocks cell proliferation by inhibiting translation, found that modulation of eIF4A can control cellular activities (Yang et al., 2003). The translational inhibition is caused by binding of Pdcd4 to eIF4A, which competitively blocks eIF4A-binding to the C-terminal domain of eIF4G and inhibits helicase activity of eIF4A (Yang et al., 2003). In this respect, the translation inhibitory of 15d-PGJ2 is likely to contribute to anti-neoplastic activity of 15d-PGJ2. These indicate that eIF4A is a good target for the regulation of biological activities intracellularly through Pdcd4 and intercellularly through 15d-PGJ2 and that eIF4A is a good therapeutic target for developing anti-cancer drugs.

The eIF4A amino acid residue targeted by 15d-PGJ2 was identified by monitoring the 15d-PGJ2-binding capabilities of mutant eIF4As (FIG. 6E), and the cysteine at residue 264 was found to be the target site of 15d-PGJ2 (FIG. 6E). Interestingly, the cysteine 264 is positioned close to the eIF4A residues R360, R363 and R366 that had previously been shown, by site-directed mutagenesis, to be important for binding to the middle and the C-terminal domains of eIF4G1 (Zakowicz et al., 2005). Moreover, cysteine 264 is next to the residues aspartic acid 265 and glutamic acid 268 that have been shown to be essential for interaction with eIF4G1 (Oberer et al., 2005). Cysteine 264 is located in the α-helix that forms a contact surface with the middle domain of eIF4GI, as shown by nuclear magnetic resonance (NMR) spectroscopy (Oberer et al., 2005). Therefore, it is possible that PGJ2 covalently bound to eIF4A at the eIF4G contact site sterically hinders the eIF4A-eIF4G interaction.

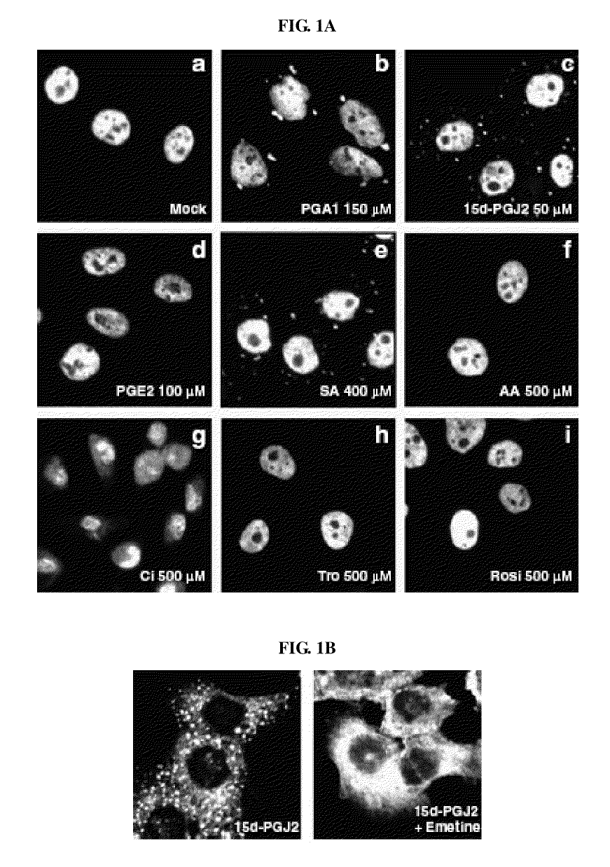

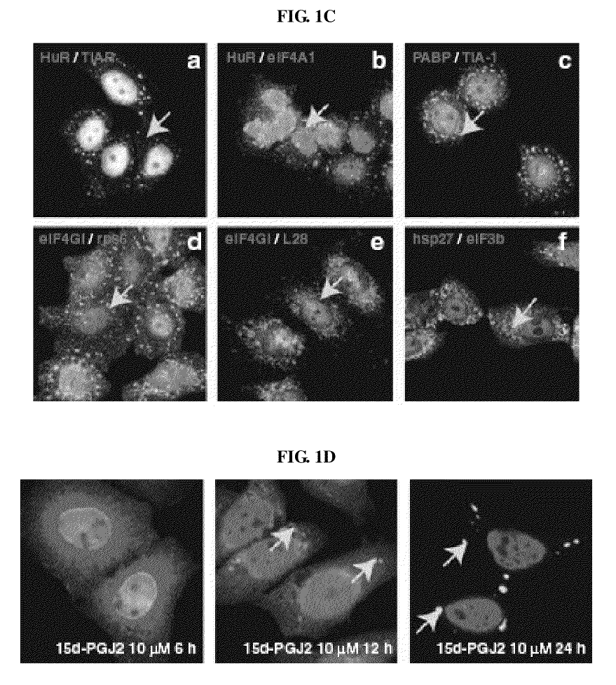

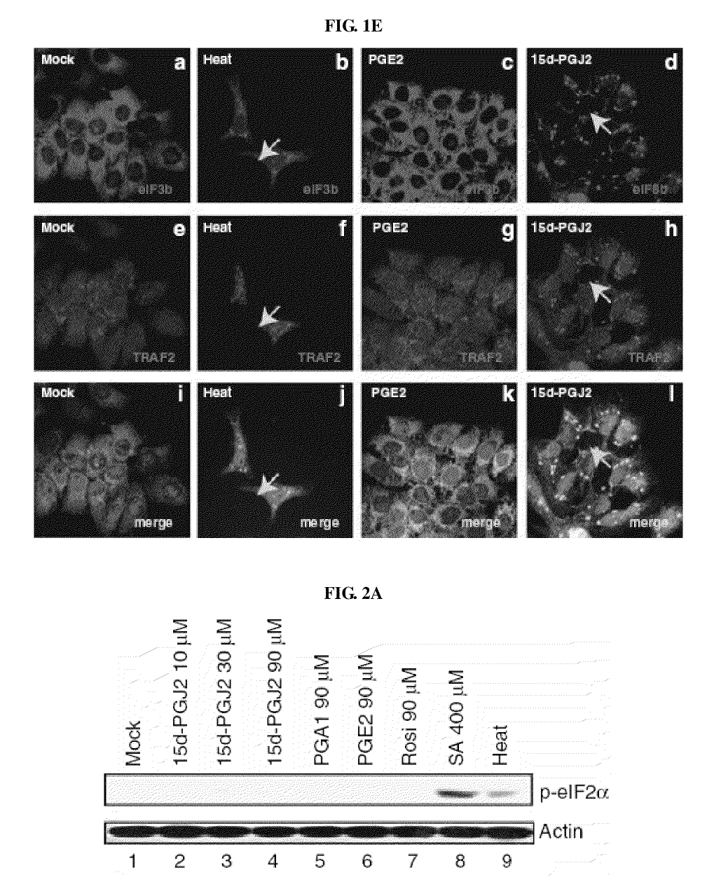

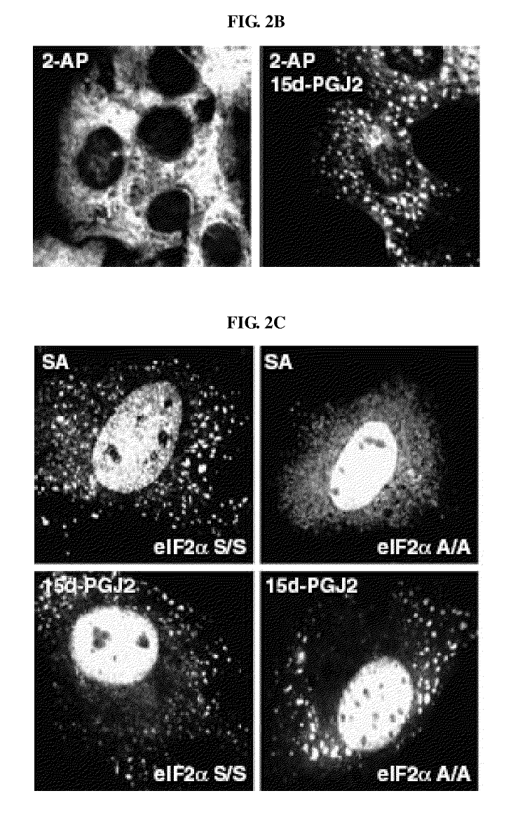

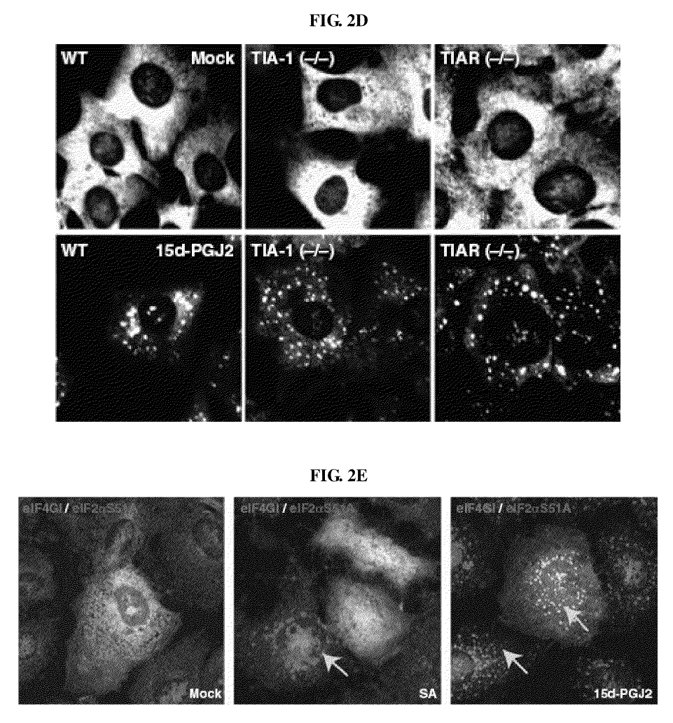

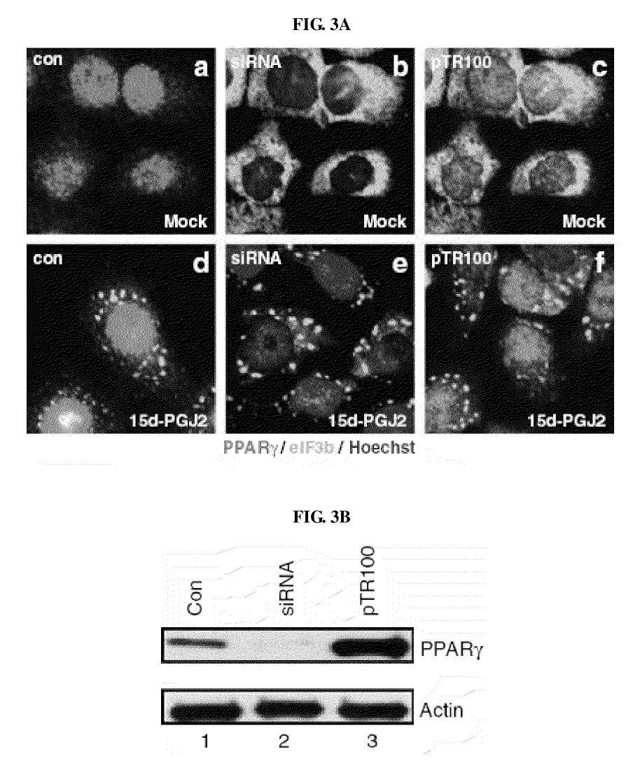

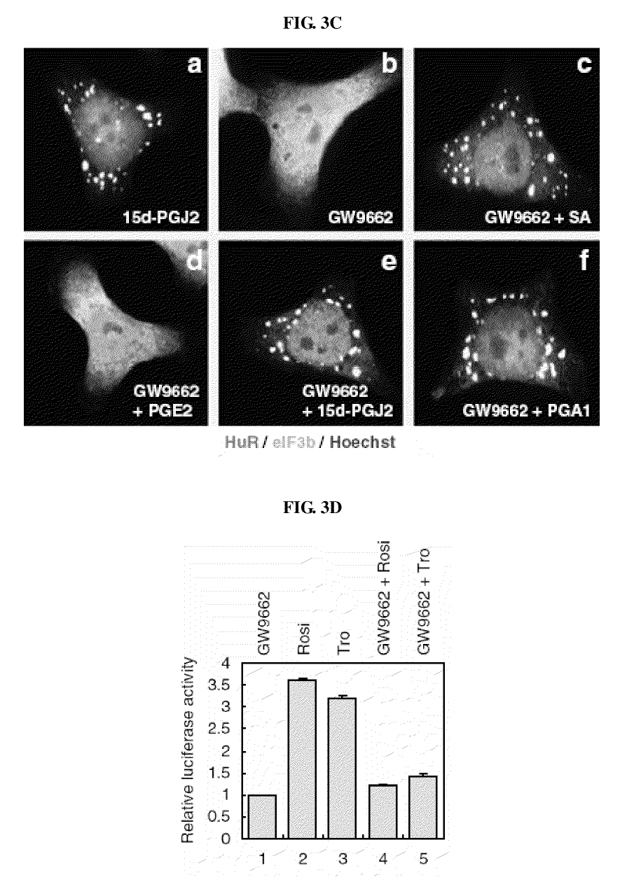

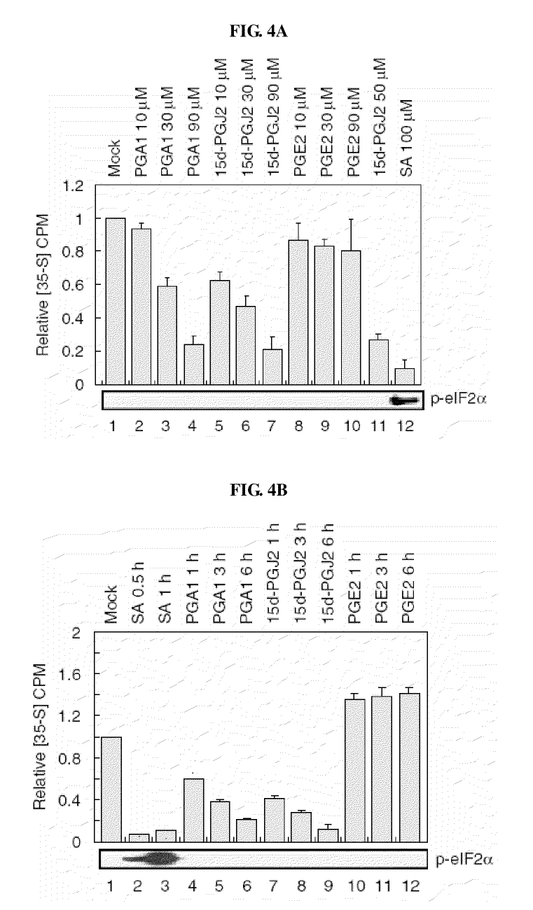

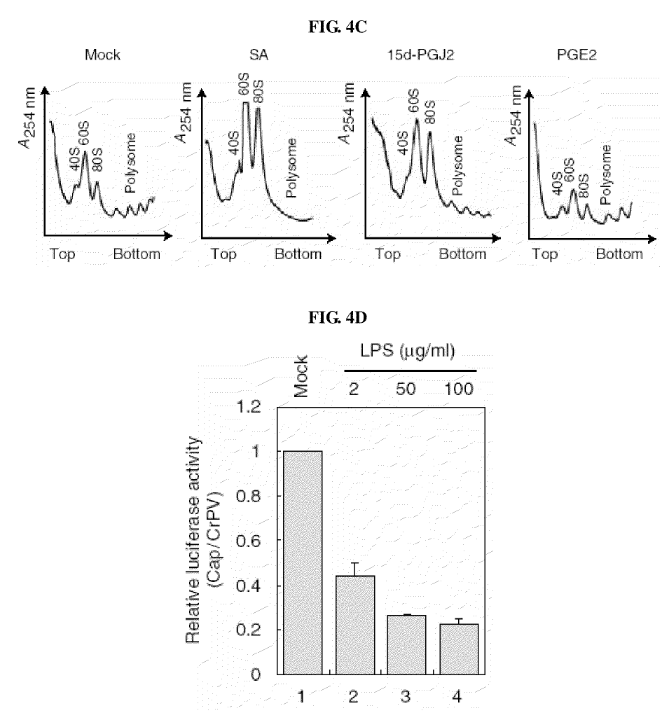

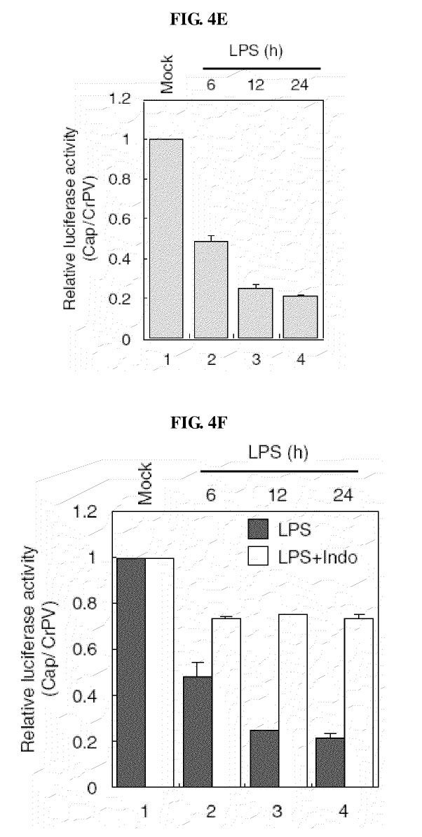

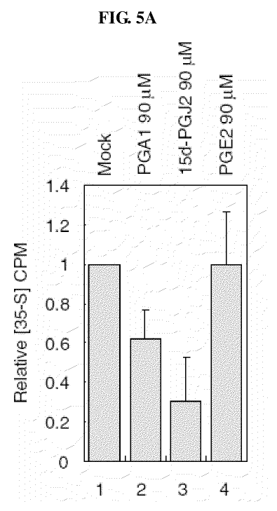

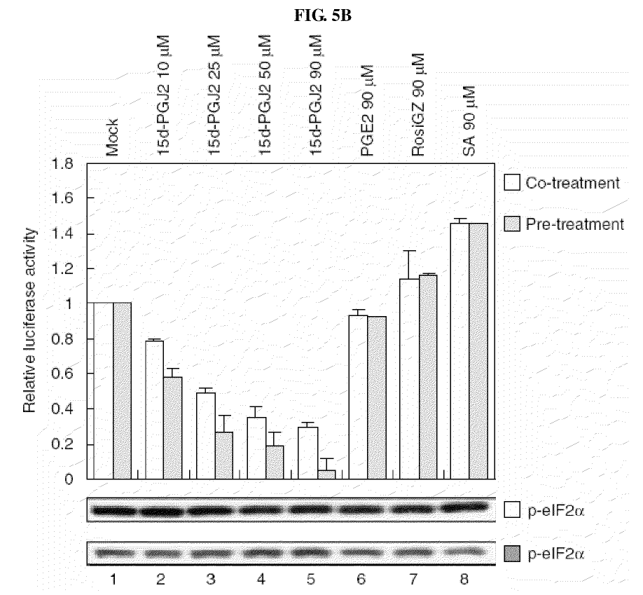

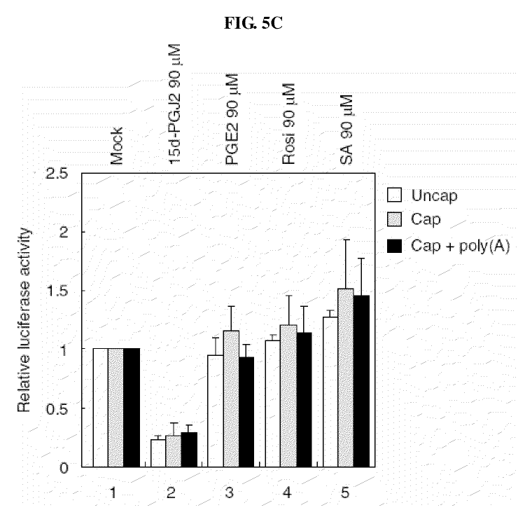

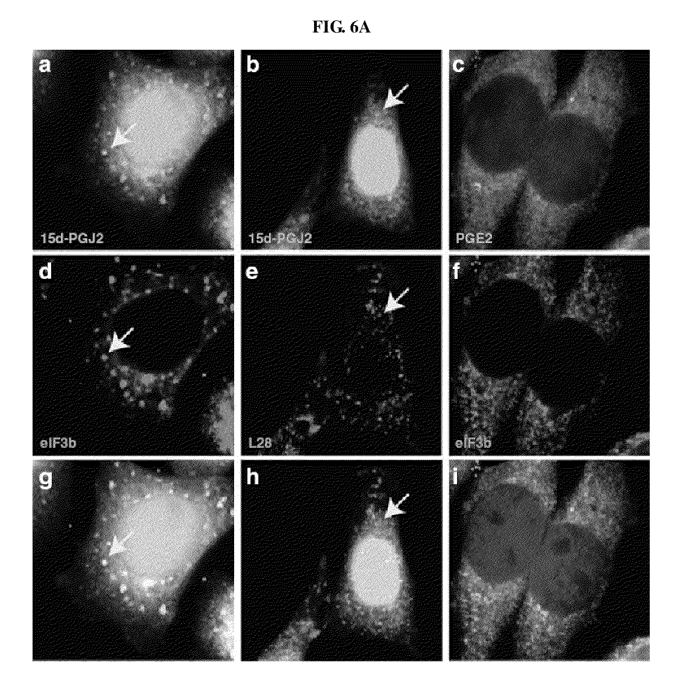

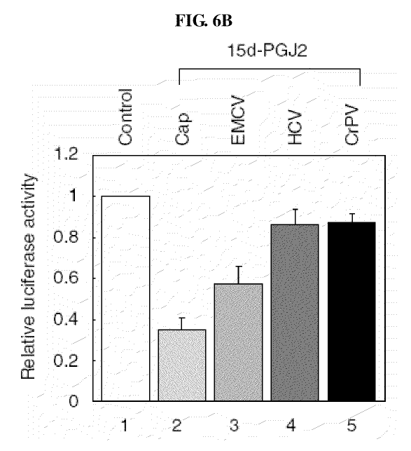

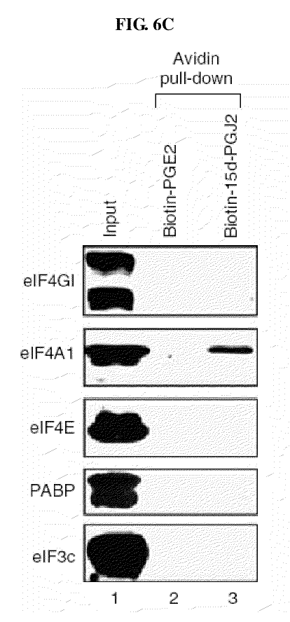

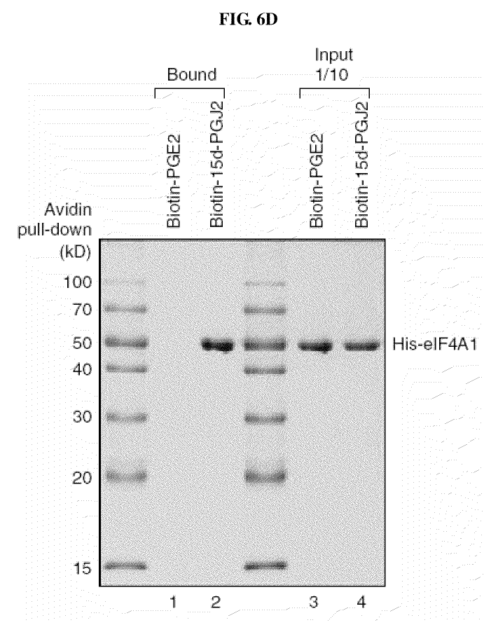

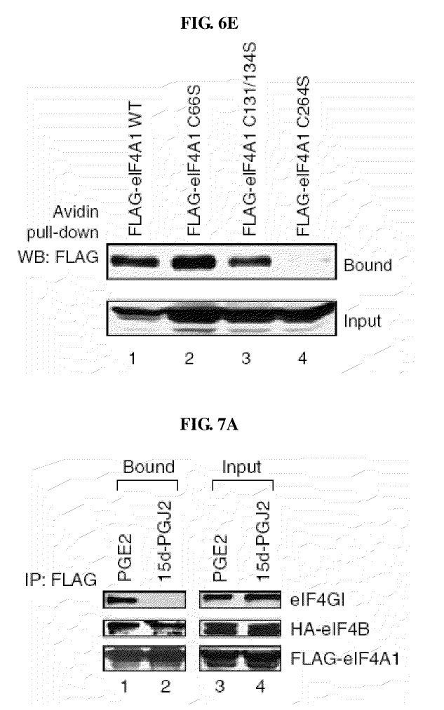

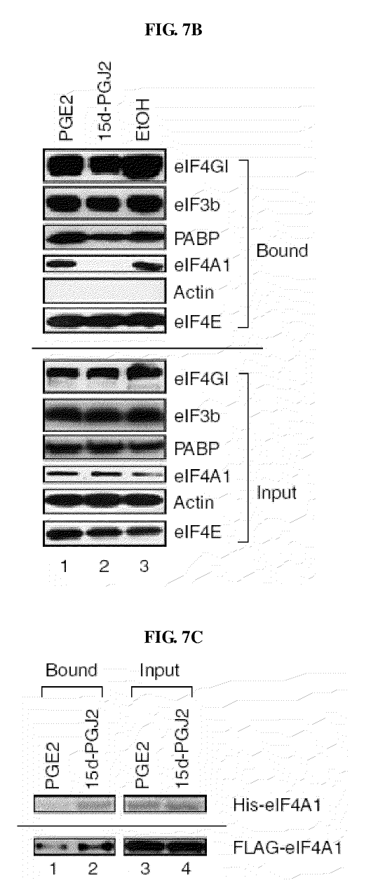

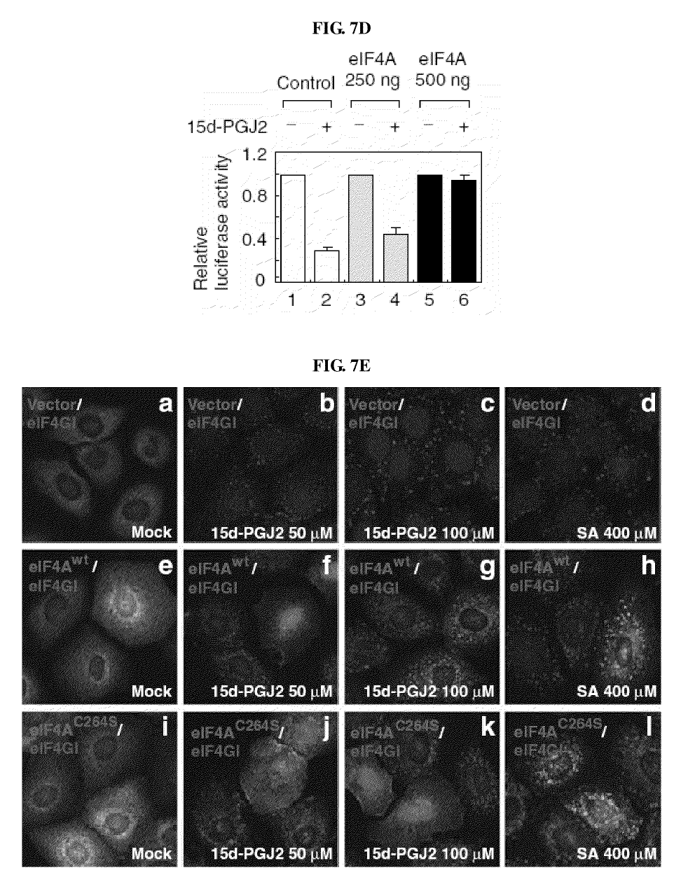

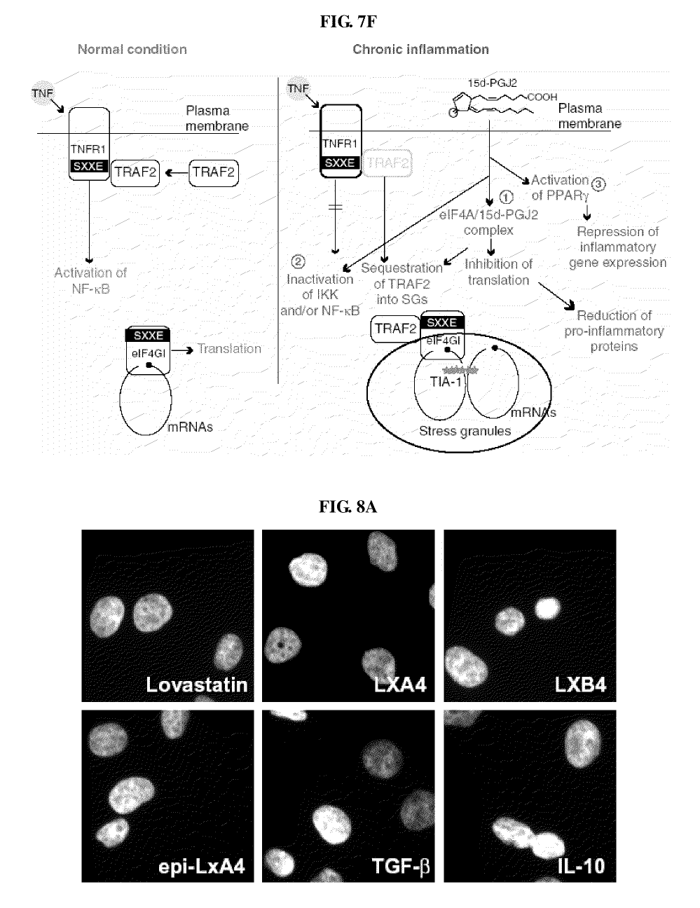

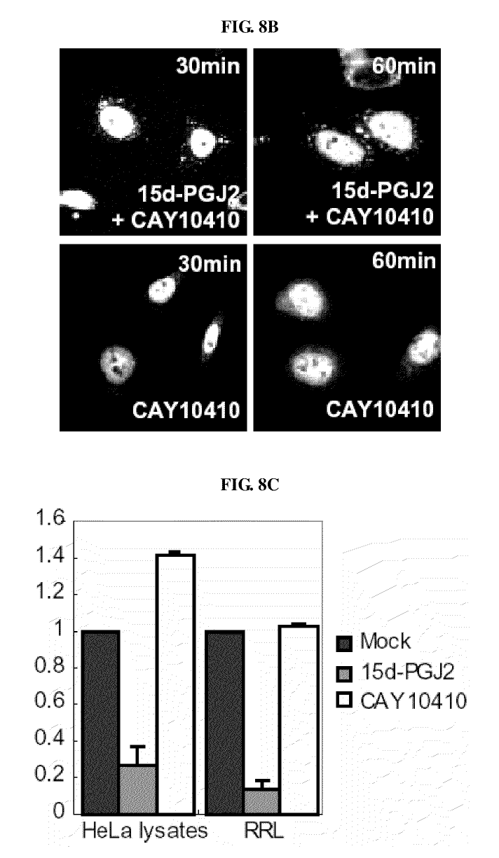

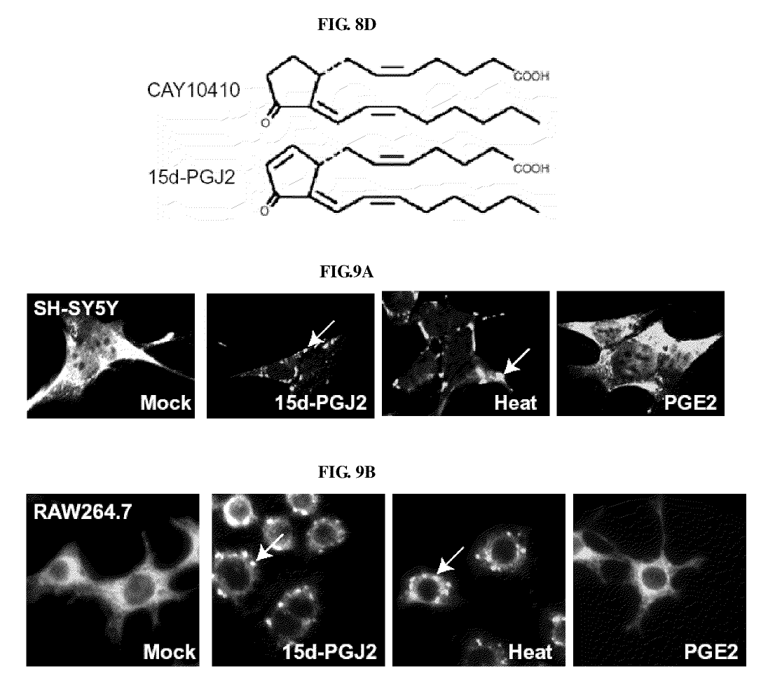

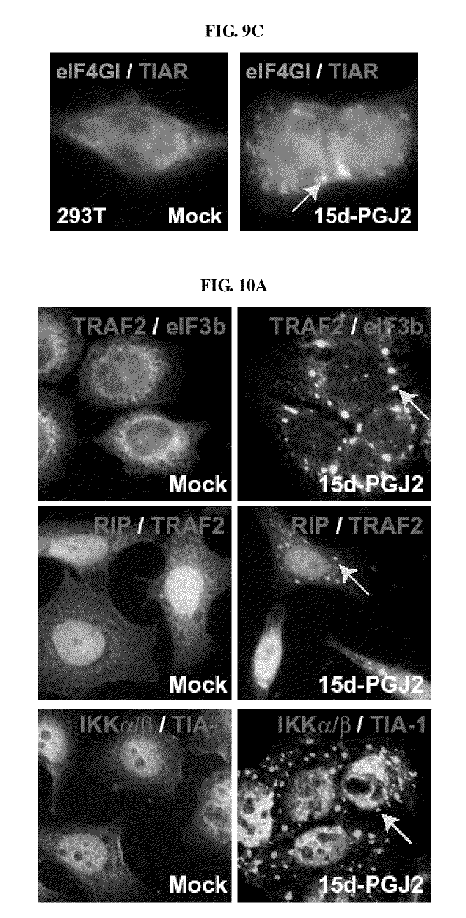

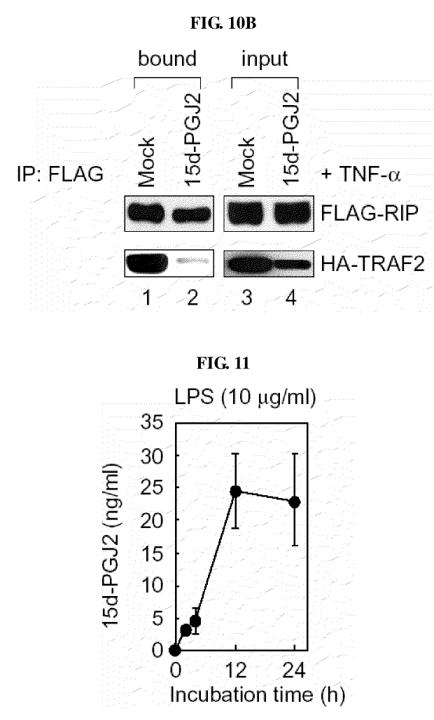

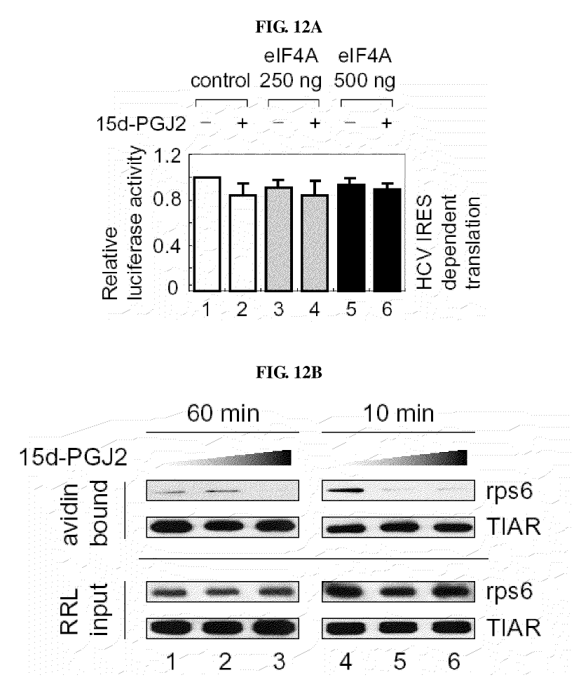

SGs are formed under various conditions that block translation. SG formation owing to 15d-PGJ2 treatment (FIG. 1) is most likely to be due to inhibition of translation by eIF4A-15d-PGJ2 complex formation. Interestingly, TRAF2 proteins, which are scaffold proteins that recruit pro-inflammatory signaling proteins, are sequestered to 15d-PGJ2-induced SGs (FIG. 1E) and heat-induced SGs (FIG. 1E; also see Kim et al., 2005) (Kim et al., 2005). Among the TNF-α signaling molecules tested, only TRAF2 was sequestered into SGs (Fig. S3A). Furthermore, RIP-TRAF2 interaction, which is required for NF-□B activation mediated by TNF-α (Cheng and Baltimore, 1996), was weakened by 15d-PGJ2 treatment (Fig. S3B). This may indicate that the sequestration of TRAF2 into SGs contributes, at least in part, to the anti-inflammatory activity of 15d-PGJ2. However, it is difficult to quantify the contribution of TRAF2 sequestration to the anti-inflammatory activity of 15d-PGJ2 because 15d-PGJ2 reduces pro-inflammatory gene expression through activation of PPARγ and inhibits TNF-α signaling by directly inactivating NF-κB and IKK (Straus et al., 2000).

Moreover, inhibition of translation by 15d-PGJ2 may also contribute to anti-inflammatory responses by lowering the levels of labile proteins required for maintaining inflammatory responses. The hypothetical process by which chronic inflammatory responses are resolved is shown in FIG. 7F. In FIG. 7F, at the chronic inflammatory stage, 15d-PGJ2 is highly produced by immune cells and inhibits the positive feedback loop of inflammation (Gilroy et al., 2004). There are multiple target molecules of 15d-PGJ2 in the cell. Modifications of some of the target proteins result in anti-inflammatory activity. Pathway 1, 15d-PGJ2 inactivates eIF4A, resulting in inhibition of translation, as described in this paper. This induces SG formation and TRAF2 proteins are sequestered into the SGs. This in turn blocks the key pro-inflammatory TNF-α signaling pathway. Translational inhibition may also reduce the expression of pro-inflammatory proteins. Pathway 2, 15d-PGJ2 directly inactivates pro-inflammatory molecules such as IKK and NF-κB (Straus et al., 2000). Pathway 3, 15d-PGJ2 functions as an agonist of PPARγ that represses transcriptional activation of inflammatory response genes. These anti-inflammatory responses may occur independently or in a concerted manner depending on the concentration of 15d-PGJ2 and on internal and external conditions of target cells.

Here, we report that 15d-PGJ2 inhibits translation by inactivating eIF4A. This activity is most likely related to the anti-proliferation activity of 15d-PGJ2. Further investigations into this activity of 15d-PGJ2 will provide clues for development of anti-cancer drugs that target eIF4A. Moreover, such research would extend our understanding of the anti-inflammatory activity of 15d-PGJ2.

The present invention is further explained in more detail with reference to the following examples. These examples, however, should not be interpreted as limiting the scope of the present invention in any manner.

### Example 1

### Example 1

### Plasmid Construction

Plasmid pcDNA3.1-FLAGX3-eIF4A1 was provided Drs. Yongjun Dang and Jian Liu in Johns Hopkins University. FLAG-eIF4A mutagenesis was performed by using overextension PCR method. To mutate the 66^{th} amino acid residue, cysteine (cysteine 66) to serine (C66S), PCR products, which were amplified using primer set GCCATTCTACCTTCTATCAAGGGTTATG (C66S forward) and CATAACCCTTGATAGAAGGTAGAATGGC (C66S reverse), were re-amplified by using BamHI and EcoRI primer set GTAGTCAGCCCGGGATCC (BamHI) and GCTTGCGGCCGCGAATTC (EcoRI). To mutate 131/134 cysteine to serine, primer set GGGCGCCTCCTCTCACGCCTCTATC (C131/134S forward) and CATACAAGTCAGATAGTGTGTCC (C131/134S reverse) were used. To mutate 264 cysteine (the 264^{th} amino acid residue, cysteine) to serine (C264S), primer set GGACACACTATCTGACTTGTATG (C264S forward) and CATACAAGTCAGATAGTGTGTCC (C264S reverse) were used.

The obtained PCR products with mutations were treated with BamHI/EcoRI (New England Biotech, NEB) and then inserted into pcDNA3.1-FLAGX3-eIF4A1 treated with BamHI/EcoRI. The clones were confirmed by DNA sequencing. To construct pRLCMV-poly(A)60, pRF-skp25′+A was treated with NotI then inserted into pRLCMV treated with NotI (NEB). To construct pRL-CrPV, pRL-CrPV dual reporter (kindly provided by Dr. Peter Sarnow in Stanford University) was treated with EcoRI/BamHI and then inserted into pSK(−) treated with EcoRI/BamHI. To construct pRL-EMCV, pRL-EMCV dual reporter was treated with XbaI/NheI/Klenow (NEB) and then self-ligated. To construct pRL-HCV, pRL-HCV dual reporter was treated with Asp718/Klenow/NotI (NEB) and then inserted into pRL-HCV dual reporter treated with NheI/Klenow/NotI (NEB). To construct pCMV-HA-eIF4B, a DNA fragment generated from pSK-eIF4B by treating with EcoRI and Klenow fragment was inserted into pCMV-HA treated with EcoRI and Klenow fragment. pcDNA3-7B-ARE-MS2bs was kindly provided by Dr. Satoshi Yamasaki at Brigham and Women's Hospital. Plasmids with a PPRE reporter (provided by Dr. Todd Leff, Wayne State University) and pcDNA3.1-PPARγ were kindly provided by Dr. Todd Leff in Wayne State University. siRNA against PPARγ corresponding to nucleotides 105-123 (5′-GCCCTTCACTACTGTTGAC-3′) was synthesized from Bioneer.

### Example 2

### Preparation of Antibodies and Chemicals

Antibodies against TIA-1, eIF3b, eIF3c, eIF4A1, eIF4E, HA, HuR, hsp70, L28, TIA-1, TIAR, PABP, PPARγ, RIP, IKKα/β, and rps6 were purchased from Santa Cruz. Antibodies against TRAF2, FLAG, actin, and hsp27 were purchased from BD Pharmingen, Sigma, ICN, StressGen, respectively. Antibodies against eIF2α and phospho-eIF2α were purchased from Cell Signaling Technology. Antibody against eIF4GI was prepared in our laboratory (Kim et al., 2005, which is hereby incorporated by reference for all purposes as if fully set forth herein).

Chemicals PGA1, 15d-PGJ2, biotinylated 15d-PGJ2, PGE2, biotinylated PGE2, arachidonic acid, ciglitazone, troglitazone, rosiglitazone, CAY10410, lipoxin A4, lipoxin B4, epi-lipoxin A4, LPS, and lovastatin were purchased from Cayman Chemical. Sodium arsenite, emetine, indomethacin, and 2-AP were purchased from Sigma. GW9662 was purchased from Calbiochem. TGF-β and IL-10 from R & D Systems.

Immobilized streptavidin agarose was purchased from Pierce. 7m-GTP-Sepharose 4B, Protein G agarose were purchased from GE Healthcare.

### Example 3

### Ribosomal Pull-Down

Ribosomal pull-downs were performed as described by Colon-Ramos et al. (Colon-Ramos et al., 2006, which is hereby incorporated by reference for all purposes as if fully set forth herein). Biotinylated β-globin mRNAs synthesized from plasmid pcDNA3-7B-ARE-MS2bs were incubated in an RRL (Promega) in the presence or absence of 15d-PGJ2. After the translation reactions, 10 μg/ml of cycloheximide was added to stop the translation, and then reaction mixtures were incubated with 50 μl of streptavidin Sepharose beads at 4° C. for 1 h. Precipitated resins were washed three times, resolved by SDS-PAGE and then transferred to a nitrocellulose membrane.

### Example 4

### Pull-Down with Streptavidin

DNA-transfected HeLa or 293T cells were lysed by soaking in NP-40 lysis buffer [0.5% NP-40, 50 mM HEPES (pH 7.4), 250 mM NaCl, 2 mM EDTA, 2 mM sodium orthovanadate, 2.5 mM β-glycerophosphate, 1 μg/ml aprotinin, 1 μg/ml antipain, 1 μg/ml bestatin, 1 μg/ml pepstatinA, 1 mM PMSF]. Lysates were clarified by centrifugation at 14,000 g at 4° C. for 15 minutes and then incubated with 50 μM of biotinylated PGE2 or biotinylated 15d-PGJ2 at 30° C. for 1 hour. After incubation, lysates were clarified by centrifugation at 14,000 g at 4° C. for 5 minutes and then incubated with 50 μl slurry of immobilized streptavidin agarose at 4° C. for 1 hour. Precipitated resins were washed three times with the lysis buffer, resolved by SDS-PAGE and then transferred to a nitrocellulose membrane.

In the 15d-PGJ2-binding experiment with purified eIF4A1 (provided by Dr. Nadejda Korneeva, Louisiana State University) and biotinylated 15d-PGJ2, 6 μg of eIF4A were used in 400 μl of the NP-40 lysis buffer.

### Example 5

### Analysis of Components of eIF4F Complex

HeLa Cells were lysed with the NP-40 lysis buffer. Cell lysates were incubated with ethanol (EtOH), PGE2, or 15d-PGJ2 at 30° C. for 1 hour. After incubation, lysates were clarified by centrifugation at 14,000 g at 4° C. for 5 minutes and then incubated with 50 μl slurry of 7m-GTP-Sepharose 4B (GE healthcare) at 4° C. for 1 hour. Precipitated proteins bound to resin were washed three times with the lysis buffer, resolved by SDS-PAGE and then transferred to a nitrocellulose membrane.

### Example 6

### Immunoprecipitation

293T cells transfected with DNAs were lysed using the NP-40 lysis buffer. The lysates were clarified by centrifugation at 14,000 g for 15 minutes. Anti-FLAG monoclonal antibody (4 μg) was incubated with 20 μl of Protein G-agarose (GE Healthcare) for 1 hour in 1 ml NP-40 lysis buffer at 4° C. Lysates were pre-cleared with 10 μl of protein G-agarose at 4° C. for 30 minutes. After pre-clearing, cell lysates were treated with 50 μM of EtOH, PGE2, or 15d-PGJ2 at 30° C. for 1 hour, followed by centrifugation. Then protein G agarose-conjugated antibodies were incubated with the pre-cleared lysates at 4° C. for 1 hour. Precipitates were washed three times with lysis buffer and analyzed by SDS-PAGE.

### Example 7

### In Vitro Transcription and Pull-Down with Biotinylated RNAs

For in vitro transcription, monocistronic reporter plasmids were digested by HpaI (NEB) and then analyzed by the T7 polymerase (NEB) reaction. pcDNA3-7B-ARE-MS2bs (kindly provided by Dr Satoshi Yamasaki, Brigham and Women's Hospital) digested by XbaI (NEB) before use in the T7 polymerase reaction in the presence of biotinylated UTP. Before incubation with the biotinylated RNAs, cell lysates were incubates with 50 μM PGE2 or 15d-PGJ2 at 30° C. for 1 hour, followed by clarification with centrifugation. RNA-affinity chromatography was performed with purified His-eIF4A or 293T cell lysates transfected with FLAG-eIF4A, as described elsewhere (Kim et al., 2004, which is hereby incorporated by reference for all purposes as if fully set forth herein).

### Example 8

### Preparation of Hela Cell Lysates and In Vitro Translation

In vitro translation reactions using HeLa cell lysates and RRL (rabbit reticulocyte lysate) are described elsewhere (Kim et al., 2004, which is hereby incorporated by reference for all purposes as if fully set forth herein).

### Example 9

### Ribosome Profiling with Sucrose Gradient

Cells were treated with various agents and for various times, as indicated in the figure legends. Experiments were performed as described elsewhere (Kedersha et al., 2000, which is hereby incorporated by reference for all purposes as if fully set forth herein) using a 0.5-1.5 M sucrose gradient.

### Example 10

### Fluorescence Microscopy

The immunocytochemical analyses of proteins were performed as described elsewhere (Kim et al., 2005, which is hereby incorporated by reference for all purposes as if fully set forth herein).

### Example 11

### Monitoring Newly Synthesized Proteins with [³⁵S]-Labeling

HeLa cells were treated with various agents and for various times, as indicated in FIG. 5A and the brief description thereof. HeLa Cells on 60-mm culture dishes were then washed twice with phosphate-buffered saline (PBS) and incubated in methionine-free Dulbecco's Modified eagle's medium (DMEM) (BMS) medium for 1 hour. Cells were incubated for 30 minutes after supplementation with [³⁵S]-methionine ([³⁵S]-Met) (500 mCi/ml; NEN Life Science Products), washed twice with ice-cold PBS, harvested, and then lysed with the NP-40 lysis buffer. The cell lysates were centrifuged and the protein concentrations in the cell lysates were measured using the Bradford assay method. To quantify newly synthesized proteins, cell lysates labeled with [³⁵S]-Met were precipitated with 10% trichloroacetic acid (TCA) (w/v), and the precipitated proteins were then dissolved in water and analyzed by a liquid scintillation assay (Packard).

### Example 12

### Luciferase Assay

Luciferase assays were performed as described elsewhere (Kim et al., 2005, which is hereby incorporated by reference for all purposes as if fully set forth herein).

### Example 13

### Cell Cultures and Transient Transfection

MEF TIA (−/−), and MEF TIAR (−/−) cells (provided by Dr. Nancy Kedersha and Paul Anderson, Brigham and Women's Hospital), and MEF eIF2α S51A cells (provided by Dr. Randal Kaufman and Dr. Sung Hoon Back, University of Michigan Medical Center) were grown as described elsewhere (Gilks et al., 2004). RAW 264.7 cells, SH-SY5Y cells, and HeLa cells and 293T cells were grown as described elsewhere (Kim et al., 2005, which is hereby incorporated by reference for all purposes as if fully set forth herein).

### Experimental Example 1

### Induction of SG Formation by Cyclopentone Prostaglandins

Pro-inflammatory signal transduction can be blocked by sequestration of TRAF2 into SGs (Kim et al., 2005, which is hereby incorporated by reference for all purposes as if fully set forth herein). This indicates that SG formation is a potential regulatory mechanism of inflammatory signaling. Therefore, the inventors tried to identify physiological compounds that induce SG formation.

Of the compounds tested, the cyPGs 15d-PGJ2 and PGA1 induced SG formation, which are shown as cytoplasmic speckles in FIG. 1A. HeLa cells that were mock-treated (Mock) or treated with PGA1, 15d-PGJ2, PGE2, SA, arachidonic acid (AA), ciglitazone (Ci), troglitazone (Tro), and rosiglitazone (Rosi) (at indicated concentrations) for 30 minutes. Then, immunocytochemical analyses were performed using TIA-1 antibody, and the results are shown in FIG. 1A. As shown in FIG. 1A, this research focused on the effect of 15d-PGJ2 because it was a stronger SG inducer than PGA1 (FIG. 1A, compare panel b with c). It should be noted that the anti-inflammatory activity of 15d-PGJ2, in either a PPARγ-dependent or -independent manner, is stronger than that of PGA1 (Straus and Glass, 2001). On the other hand, arachidonic acid (FIG. 1A, panel f), which is the precursor of cyPGs, and a pro-inflammatory PG PGE2 (FIG. 1A, panel d) did not induce SG formation. Moreover, the PPARγ agonists ciglitazone, troglitazone, and rosiglitazone did not induce SG formation (FIG. 1A, panels g-i).

HeLa cells were treated with lovastatin (100 μM), lipoxin A4 (LXA4, 10 μM), lipoxin B4 (LXB4, 10 μM), epi-lipoxin A4 (epi-LxA4, 10 μM), TGF-β (100 ng/ml), interleukin-10 (IL-10, 100 ng/ml) for 1 hour. Immunocytochemical analyses were performed with a TIA-1 antibody and the obtained results are shown in FIG. 8A. SG formation was not observed from cells treated with most of anti-inflammatory compounds tested.

In addition, CAY10410, an analogue of 15d-PGJ2, does not induce SG formation. HeLa cells grown on cover slips were treated with CAY10410 (50 μM) alone (lower panels) or together with 15d-PGJ2 (upper panels) for 30 minutes (left panels) or 60 minutes (right panels), respectively. Immunocytochemical analyses were performed with a TIA-1 antibody and the obtained results are shown in FIG. 8B.

CAY10410 does not inhibit translation. In vitro translation reactions were performed with a Renilla luciferase mRNA (40 nM) in HeLa lysates and RRL (rabbit reticulocyte lysate) in the presence of vehicle (black columns), 50 μM 15d-PGJ2 (gray columns), or CAY10410 (white columns). Relative luciferase activities are shown in FIG. 8C, with mock-treated translation reactions set to 1. The bars indicate standard deviation values of three independent experiments.

The chemical structures of 15d-PGJ2 and CAY10410 are shown in FIG. 8D. CAY10410 is an analog of prostaglandin D2/prostaglandin J2 (PGD2/PGJ2) with structural modifications that are intended to maintaining PPARγ agonist activity and resistance to metabolism.

The identity of the cytoplasmic speckles was confirmed by emetine treatment. Emetine freezes ribosomes in the polysomal state and inhibits SG formation (Anderson and Kedersha, 2006). HeLa cells that were pretreated for 1 hour with 10 μg/ml of emetine and then treated with 15d-PGJ2 (50 μM) for 1 hour, and subjected to immunocytochemical analyses. The obtained results are shown in FIG. 1B. As shown in FIG. 1B, emetine treatment inhibits 15d-PGJ2-induced SG formation (right panel) in the same manner as it inhibits those induced by sodium arsenite (SA) or heat. This clearly demonstrates that 15d-PGJ2-induced SGs share similar properties to the typical SGs induced by heat or SA.

The components of SGs induced by 15d-PGJ2 were analyzed using an immunocytochemical method. HeLa cells were treated with 15d-PGJ2 (50 μM) for 1 hour. Immunocytochemical analyses were performed with the indicated antibodies: HuR/TIAR (a), HuR/eIF4A1 (b), PABP/TIA-1 (c), eIF4GI/rps6 (d), eIF4GI/L28 (e), and hsp27/eIF3b (f). The obtained results are shown in FIG. 1C, wherein nuclei are shown in blue by Hoechst staining and arrows indicate SGs. As expected, known SG marker proteins (TIA-1 and TIAR), an RNA-binding protein (HuR), translational initiation factors [eIF4GI, eIF3b, and poly(A)-binding protein (PABP)], and the 40S ribosomal subunit (as indicated by the rps6 ribosomal protein) were observed in the SGs (FIG. 1C). By contrast, the 60S ribosomal subunit, as indicated by the ribosomal protein L28, was not localized to the SGs induced by 15d-PGJ2 (FIG. 1C, panel e). Interestingly, heat-shock protein 27 (hsp27), which is localized in the SGs induced by heat but not in the SGs induced by SA (Anderson and Kedersha, 2006), was enriched in the SGs induced by 15d-PGJ2 (FIG. 1C, panel f).

The amounts of 15d-PGJ2 required for SG formation were measured. HeLa cells were treated with 15d-PGJ2 (10 μM) for 12-24 hours indicated in FIG. 1D. Immunocytochemistry was performed with an eIF3b (green) and HuR (red) antibodies, and the results are shown in FIG. 1D, wherein arrows indicate SGs. SGs were formed by 10 μM of 15d-PGJ2 after 12-24 hours of treatment, as shown in FIG. 1D. Anti-inflammatory response (Campo et al., 2002; Straus et al., 2000) and other biological activities (Aldini et al., 2007; Arnold et al., 2007; Fionda et al., 2007; Hasegawa et al., 2007; Lin et al., 2007; Pereira et al., 2006) of 15d-PGJ2 were observed at these conditions.

As shown in FIG. 1E, HeLa cells were treated with heat at 44° C. (b, f, and j), 50 μM of PGE2 (c, g, and k), or 15d-PGJ2 (d, h, and 1) for 1 hour. Immunocytochemistry was performed using eIF3b and TRAF2 antibodies, wherein arrows indicate SGs. Subcellular localizations of TRAF2 before (FIG. 1E, panels a, e and i) and after induction of SG formation by heat (FIG. 1E, panels b, f and j) or 15d-PGJ2 (FIG. 1E, panels d, h and l) were monitored by an immunocytochemical method. This was because the sequestration of TRAF2 into SGs induced by heat has previously been reported (Kim et al., 2005). Similarly, migration of TRAF2 to SGs induced by 15d-PGJ2 was observed, as indicated by co-localization with eIF3b (yellow dots in FIG. 1E, panel l). There was no change in the subcellular localization of TRAF2 with PGE2 treatment (FIG. 1E, panels c, g and k).

Furthermore, we found that 15d-PGJ2 induced SG formation in various cell lines such as a neuronal cell line SH-SY5Y and a macrophage cell line RAW264.7 (Fig. S2). In subsequent experiments, we treated HeLa cells with 50 μM 15d-PGJ2 for 1 hour to induce SGs quickly, unless otherwise indicated.

SH-SY5Y cells originated from a human neuroblastoma were mock-treated or treated with 15d-PGJ2 (30 μM) for 1 h, heat at 44° C. for 1 h, or PGE2 (30 μM) for 1 h. Immunocytochemical analyses were performed with antibodies against eIF3b, and the results are shown in FIG. 9A. The same set of experiments as outlined in FIG. 9A were performed with RAW264.7 macrophage cells. Immunocytochemical analyses were performed with antibodies against eIF3b and the obtained results are shown in FIG. 9B. HEK 293T cells were mock-treated or treated with 15d-PGJ2 (50 μM) for 1 hour. Immunocytochemical analyses were performed with antibodies against eIF4GI and TIAR and the obtained results are shown in FIG. 9C.

The inventors also investigated the localization of RIP, which directly interacts with TRAF2 and conveys TNF-α signal downstream of TRAF2 (Jackson-Bernitsas et al., 2007), and that of IKK α/β which conveys TNF-α signal downstream of RIP and is also known as a target of 15d-PGJ2 (Cheng and Baltimore, 1996). Neither RIP nor IKK α/β was sequestered into SGs (FIG. 10A). In FIG. 10A, HeLa cells were mock-treated (left panels) or treated with 50 μM of 15d-PGJ2 (right panels) for 1 hour. Immunocytochemical analyses were performed with the indicated antibodies: TRAF2 and eIF3b (upper panels), RIP and TRAF2 (middle panels), IKKα/β and TIA-1 (lower panels). Arrows indicate SGs. Similar phenomenon was observed in the SGs induced by heat stress (Kim et al., 2005).

Moreover, the interaction between RIP and TRAF2 was inhibited by 15d-PGJ2 treatment (FIG. 10B). In FIG. 10B, 293T cells were co-transfected with plasmids expressing FLAG-tagged RIP and HA-tagged TRAF2. After 48 hours of incubation, cells were mock-treated or treated with 50 μM of 15d-PGJ2 for 1 hour and then treated with 100 ng/ml of TNF-α for 30 min. Immunoprecipitation was performed with an anti-FLAG antibody. Western-blot analyses were performed with anti-FLAG and anti-HA antibodies. These results indicate that the sequestration of TRAF2 by 15d-PGJ2 contributes to the anti-inflammatory activity of this lipid molecule independently of inactivation of IKK and NF-KB by this compound (Straus et al., 2000).

### Experimental Example 2

### SG formation by 15d-PGJ2

In this example, it was confirmed that SG formation by 15d-PGJ2 does not need eIF2α phosphorylation, TIA-1 aggregation, and PPARγ activation

To understand the molecular basis of 15d-PGJ2-induced SG formation, eIF2α phosphorylation levels was assessed by using a phospho-eIF2α-specific antibody, because some SG-inducing agents such as SA induce SG formation by phosphorylation of eIF2α (Anderson and Kedersha, 2006).

Phosphorylated eIF2α levels were monitored by Western-blot analyses using HeLa cell extracts (40 μg) treated with 15d-PGJ2 (lanes 2-4), PGA1 (5), PGE2 (6), Rosi (7), or SA (8) at the indicated concentrations for 30 minutes or with heat at 44° C. for 30 minutes, and the obtained results are shown in FIG. 2A. As shown in FIG. 2A, there was no significant increase in eIF2α phosphorylation in the cells treated with either 15d-PGJ2 or PGA1 (FIG. 2A, lanes 2-5), although SA-treated and heat-treated cells showed increased levels of phosphorylated eIF2α (lanes 8 and 9).

The effect of 2-aminopurine (2-AP), a strong PKR (protein kinase, interferon-inducible double stranded RNA dependent activator) inhibitor, on blockade of SG formation by 15d-PGJ2 was also tested. HeLa cells grown on cover slips were pretreated with 1 mM of 2-AP or with vehicle for 6 hours, and then treated with 50 μM of 15d-PGJ2 for 30 minutes. Fixed cells were analyzed by immunocytochemistry with an eIF3b antibody, and the obtained results are shown in FIG. 2B. As shown in FIG. 2B, pretreatment with 2-AP had no effect on 15d-PGJ2-induced SG formation (right panel).

Furthermore, the wild-type and eIF2α A/A mutant MEF cells were treated with 400 μM of SA for 30 minutes or 50 μM of 15d-dPJ2 for 1 hour. Immunocytochemical assays were performed with a TIA-1 antibody, and the results are shown in FIG. 2C. As shown in FIG. 2C, 15d-PGJ2 induced SG formation in a MEF cell with a mutant eIF2α (eIF2α A/A cell) with a S51A knock-in mutation at the PKR target site of the eIF2α gene (McEwen et al., 2005) (FIG. 2C, bottom panels). On the other hand, SA-induced SG formation was inhibited in this cell line as reported (McEwen et al., 2005) (FIG. 2C, top panels).

Furthermore, a plasmid encoding FLAG tagged-eIF2α S51A was transfected into HeLa cells. After 48 hours of incubation, cells were mock-treated (left), treated with 400 μM of SA (middle) or with 50 μM of 15d-PGJ2 (right) for 30 minutes. The loci of eIF4GI and eIF2α S51A were visualized by an immunocytochemical method using eIF4GI and FLAG antibodies, respectively, and the obtained results are shown in FIG. 2E. As shown in FIG. 2E, overproduction of S51A mutant eIF2α inhibited SG formation induced by SA treatment (middle panel in FIG. 2E) as reported (Anderson and Kedersha, 2006). On the other hand, overproduction of the mutant eIF2α did not block SG formation by 15d-PGJ2 (right panel in FIG. 2E). These results suggest that phosphorylation of eIF2α is not essential for SG formation by 15d-PGJ2. These results are contradictory to a previous report suggesting that 15d-PGJ2 induces phosphorylation of eIF2α through the PKR mediated pathway (Campo et al., 2002). The discrepancy may be attributed to the difference in conditions and cell lines used in the experiments.

The prion-like activity of TIA-1 has been reported to function in SG formation (Gilks et al., 2004). The effects of TIA-1 and TIAR on 15d-PGJ2-induced SG formation were investigated using TIA-1 and TIAR KO MEF cell lines (provided by Dr. Nancy Kedersha and Paul Anderson, Brigham and Women's Hospital) as shown in FIG. 2D. The wild-type, TIA-1 KO, and TIAR KO MEF cells were mock-treated (upper panel) or treated with 15d-PGJ2 (lower panel). Immunocytochemical analyses were performed with an eIF3b antibody, and the obtained results are shown in FIG. 2D. As shown in FIG. 2D, the number of 15d-PGJ2-induced SGs was not reduced in TIA-1 KO cell line (FIG. 2D, bottom panels), unlike the level of SGs induced by other agents such as SA (Gilks et al., 2004) (data not shown). This indicates that neither TIA-1 nor TIAR has a key role in 15d-PGJ2-induced SG formation.

The role of PPARγ in 15d-PGJ2-induced SG formation was investigated because PPARγ is the best known target molecule of 15d-PGJ2 (Straus and Glass, 2001). The investigation revealed that SG formation by 15d-PGJ2 is independent of PPARγ as shown in FIGS. 3A-3D. PPARγ clones were provided by Dr. Todd Leff (Wayne State University).

HeLa cells grown on cover slips were transfected with a siRNA against PPARγ (FIG. 3A, b and e) or a plasmid pTR100-PPARγ expressing high levels of PPARγ (FIG. 3A, c and f). After transfection, cells were treated with 50 μM of 15d-PGJ2 for 1 hour. Immunocytochemical analyses were performed with eIF3b and PPARγ antibodies, shown in green and red, respectively, and the obtained results are shown in FIG. 3A. The amounts of PPARγ in cells transfected with control siRNA (FIG. 3B, lane 1), siRNA against PPARγ (FIG. 3B, lane 2) and pTR100-PPARγ (FIG. 3B, lane 3) were analyzed by Western-blot assays using a PPARγ antibody. Lysates were normalized by an actin blot. The obtained results are shown in FIG. 3B. As shown in FIGS. 3A and 3B, knock-down of PPARγ by a PPARγ-specific siRNA and overexpression of PPARγ (FIG. 3B, lanes 2 and 3) had no effect on the SG formation induced by 15d-PGJ2 (FIG. 3A, panels e and f).

HeLa cells that were pretreated with 1 μM of GW9662, an irreversible PPARγ antagonist, for 24 hours and then treated with SA (400 μM), PGE2 (50 μM), 15d-PGJ2 (50 μM), or PGA1 (50 μM) for 1 hour. Immunocytochemical analyses were performed with eIF3b and HuR antibodies, shown in green and red, respectively, and the results are shown in FIG. 3C, wherein the nuclei are shown in blue by Hoechst staining. As shown in FIG. 3C, the PPARγ-specific antagonist GW9662 also had no effect on SG formation induced by 15d-PGJ2 and PGA1 (FIG. 3C, panels e and f).

293T cells that were transfected with a plasmid (1 μg) expressing a PPARγ reporter gene. After transfection, cells were pretreated or not pretreated with 1 μM of GW9662 for 24 hours, before being treated with 10 μM of rosiglitazone (Rosi) or troglitazone (Tro) for 12 hours. The obtained relative luciferase activities in the cell extracts normalized to a mock-treated control extract are shown in FIG. 3D. As shown in FIG. 3D, under the same conditions, PPARγ-mediated PPRE (PPAR-responsive element) activation was completely blocked by GW9662 (FIG. 3D, lanes 4 and 5). These data indicate that PPARγ is not involved in 15d-PGJ2-mediated SG formation.

### Experimental Example 3

### Translation Inhibition by 15d-PGJ2

As SG formation is accompanied by translational blockade, the effects of 15d-PGJ2 on protein synthesis were investigated. In FIG. 4A, HeLa cells grown on 60-mm dishes up to about 70-80% confluence were mock-treated (1) or treated with PGA1 (2, 3, and 4), 15d-PGJ2 (5 and 6 and 7), or PGE2 (8, 9, and 10) at the indicated concentrations in FIG. 4A for 30 minutes. Then in vivo labeling of newly synthesized proteins was performed as described above. The obtained results are shown in FIG. 4A. 4200 CPM was obtained from the TCA-precipitated control sample (lane 1), and phosphorylated eIF2α levels were monitored by Western-blot analyses (bottom panel). In FIG. 4B, cells were mock-treated (1), treated with SA (400 μM) (2 and 3), with PGA1 (90 μM) (4-6), with 15d-PGJ2 (90 μM) (7-9), and with PGE2 (90 μM) (10-12) as indicated times. Newly synthesized proteins were measured as panel (A). 4500 CPM was obtained from the TCA-precipitated control sample (lane 1). Phosphorylated eIF2α levels were monitored by Western-blot analyses (bottom panel) and the obtained results are shown in FIG. 4B.

As shown in FIGS. 4A and 4B, metabolic labeling of HeLa cells with ³⁵S-methionine clearly showed that total protein synthesis was inhibited by 15d-PGJ2 in a concentration-dependent manner (FIG. 4A, lanes 5-7) and a time-dependent manner (FIG. 4B, lanes 7-9). PGA1 had a similar effect as 15d-PGJ2 (FIG. 4A, lanes 2-4; FIG. 4B lanes 4-6), but PGE2 did not block translation (FIG. 4A, lanes 8-10; FIG. 4B, lanes 10-12). No significant phosphorylation of eIF2α was observed from the cells treated with 15d-PGJ2 (FIGS. 4A and B, bottom panels).

It is well known that inhibition of translation and SG formation alters the polysome profile. HeLa cells were mock-treated or treated with SA (400 μM) for 30 minutes, 15d-PGJ2 (50 μM) for 1 hour, or PGE2 (50 μM) for 1 hour. A sucrose gradient experiment was performed as described above, and the obtained results are shown in FIG. 4C, wherein the lines show observance at 254 nm. FIG. 4C reveals that SA treatment induces the disassembly of polysomes, leading to an increase in the extents of ribosomal subunit peaks, indicating the accumulation of ribosomal subunits not participating in translation (FIG. 4C, panel SA) (Anderson and Kedersha, 2006). Ribosomal shift to the subunit state was also observed in 15d-PGJ2-treated cells, albeit the magnitude of which was weaker than that seen in SA-treated cells (FIG. 4C, panel 15d-PGJ2). As expected, PGE2 did not induce a ribosomal shift (FIG. 4C, panel PGE2). SA- and 15d-PGJ2-induced monosome shifts disappeared when cells were pretreated with emetine. These data indicate that 15d-PGJ2, similarly to SA, inhibits protein synthesis in vivo.

In order to confirm that the translational inhibition by 15d-PGJ2 occurs at physiological conditions, we tested the effect of prolonged treatment of lipopolysaccharide (LPS) on RAW264.7 that produces PGD2 and 15d-PGJ2 upon treatment of LPS through a COX-2-dependent pathway (Shibata et al., 2002). In FIGS. 4D-4F, the effects of LPS on translation in RAW264.7 macrophage cells are shown. RAW264.7 cells were incubated with LPS for 24 hours at the indicated concentrations. After the LPS treatment, mRNAs (1 μg) containing Renilla luciferase translated in a cap-dependent manner and mRNAs (1 μg) containing firefly luciferase under the control of cricket paralysis virus (CrPV) IRES were co-transfected into the cells. Luciferase activities were measured 3 hours post-transfection, and the obtained results are shown in FIG. 4D, wherein columns indicate ratios of relative luciferase activities (Renilla luciferase/Firefly luciferase) in the cell extracts normalized to that in a mock-treated control extract.

Moreover, firefly luciferase activities are considered as an indicator of mRNA transfection efficiency since CrPV IRES function is insensitive to 15d-PGJ2 as described in FIG. 6B. Monocistronic mRNAs with cap-structure (1 and 2), EMCV IRES (1 and 3), HCV IRES (1 and 4), and CrPV IRES (provided by Dr. Peter Sarnow, Stanford University) (1 and 5) were translated in HeLa lysates for 1 hour in the presence (2-4) or absence (1) of 15d-PGJ2 (50 μM). Various IRES activities (RLUs of 20,000˜75,000) were observed from the mock-treated HeLa lysates. Luciferase activities in the translation mixtures containing 15d-PGJ2 were normalized to those in the corresponding translation mixtures without 15d-PGJ2, and the obtained results are shown in FIG. 6B as columns (mean values).

Further, RAW264.7 cells were incubated with LPS (10 μg/ml) for the times indicated. Transfection of mRNAs and analyses of luciferase activities were performed as described in FIG. 4D, and the obtained results are shown in FIG. 4E.

As shown in FIGS. 4D, 4E and 6B, cap-dependent translation, but not CrPV IRES-dependent translation, was inhibited in a dose-dependent (FIG. 4D and FIG. 6B) and a time-dependent manner (FIG. 4E). The kinetics of time-dependent translational inhibition (FIG. 4E) was similar to that of 15d-PGJ2 production by LPS treatment (FIG. 11).

Moreover, in FIG. 4F, RAW 264.7 cells were pretreated (white columns) or not pretreated (grey columns) with indomethacin (1 μM) for 30 min before being treated with LPS (10 μg/ml). Transfection of mRNAs and analyses of luciferase activities were performed as described in FIG. 4D, and the obtained results are shown in FIG. 4F. FIG. 4F reveals that the translational inhibition by LPS was greatly weakened by a pretreatment of indomethacin, a non-selective COX inhibitor (compare white columns with grey columns in FIG. 4F). The effect of indomethacin treatment on translational inhibition induced by LPS is most likely attributed to blockage of 15d-PGJ2 production (Chang et al., 2006). These results suggest that translation inhibition by 15d-PGJ2 occurs at physiological conditions.

In FIG. 5A, β-gal mRNA (40 nM) was translated in HeLa lysates for 1 h in the presence of PGA1 (2), 15d-PGJ2 (3) and PGE2 (4) at indicated concentrations. [³⁵S]-labeling experiment was performed as described by Pestova et al. (Pestova et al., 1998, which is hereby incorporated by reference for all purposes as if fully set forth herein). The obtained results are shown in FIG. 5A. FIG. 5A reveals that the effect of 15d-PGJ2 on translation was also monitored using a HeLa lysate in vitro translation system. PGA1 and 15d-PGJ2 inhibited protein synthesis in vitro (FIG. 5A, lanes 2 and 3) but PGE2 did not (FIG. 5A, lane 4).

In FIG. 5B, HeLa lysates that were pretreated with vehicle (1) or with indicated chemicals (2-9) for 30 minutes at indicated concentrations, a capped Renilla luciferase mRNA (40 nM) was added to the translation mixtures, and then incubated at 30° C. for 1 hour (grey columns). White columns show the effects of the same set of chemicals added together with the reporter mRNA during 1 hour in vitro translation. Relative luciferase activities (mean values) are depicted by columns. Phosphorylated eIF2α levels were monitored by Western-blot analyses (bottom panel). FIG. 5B reveals that Pretreatment of HeLa cell lysates with 15d-PGJ2 increased the inhibitory effect about two fold with 50 μM and about five fold with 90 μM (FIG. 5B, lanes 4 and 5). Treatment of PGE2 did not block translation (FIG. 5B, lane 6). Treatment with rosiglitazone (RosiGZ) and SA slightly increased translation in vitro (FIG. 5B, lanes 7 and 8). The molecular basis of this phenomenon remains to be determined.

Basal levels of phosphorylated eIF2α were observed in HeLa cell lysates (FIG. 5B, lane 1). No increase of eIF2α phosphorylation was observed from the HeLa cell lysates treated with 15d-PGJ2, PGE2, rosiglitazone, and SA (FIG. 5B, lanes 2-8). These results indicate that eIF2α phosphorylation does not occur in HeLa cell extracts even with SA treatment (FIG. 5B, lane 8). This would be the reason why translation is not inhibited by SA in the HeLa cell lysates (FIG. 5B, lane 8) unlike in the in vivo system where eIF2α is phosphorylated by SA (FIG. 4A, lane 12).

Poly(A)-tailed mRNAs were produced by in vitro transcription of plasmid pRLCMV-poly(A)60 as described above. Capped mRNAs were produced by in vitro transcription of plasmids pRLCMV and pRLCMV-poly(A)60 in the presence of ⁷methyl GTP. In FIG. 5C, in vitro translation reactions were performed with various reporter mRNAs (40 nM) for 1 hour in the presence of chemicals (90 μM) indicated on top of the panel. Luciferase activities in the translation mixtures containing various compounds were normalized to those in mock-treated extracts with the corresponding mRNAs, and are shown as columns (mean values). Capping and poly(A) addition to the reporter mRNA did not affect the relative inhibitory activity of 15d-PGJ2 on uncapped and poly(A)-tail-less mRNA, even though the translational efficiency of capped and poly(A)-tailed mRNAs was greater than that of the uncapped tail-less mRNA (FIG. 5C, lane 2). This indicates that the eukaryotic initiation factor 4E (eIF4E), which is a cap-binding protein, or the poly(A)-binding protein (PABP) may not be involved in the translation inhibition activity of 15d-PGJ2. Pretreatment with 15d-PGJ2 increased the inhibitory effect on translation, so we speculated that a thiol modification of the target protein by the electrophilic carbon of 15d-PGJ2 is involved in the translational inhibition of 15d-PGJ2.

In addition, effects of 15d-PGJ2 on HCV IRES-dependent translation and ribosome binding of mRNA are examined, as shown in FIGS. 12A and 12B. In FIG. 12A, HCV IRES-dependent translation is not affected by 15d-PGJ2. In vitro translation of an mRNA (40 nM) containing Renilla luciferase under the control of the HCV IRES was performed in RRL (Promega) for 1 hour with the addition of purified eIF4A1 protein (0 ng, lanes 1 and 2; 250 ng, lanes 3 and 4; 500 ng, lanes 5 and 6). 15d-PGJ2 (50 μM) was added to the translation mixtures shown in lanes 2, 4, and 6. Relative luciferase activities to the mock-treated sample (lane 1) are shown in columns (mean values). Bars indicate standard deviation values. The data indicate that HCV IRES-dependent translation is affected by neither 15d-PGJ2 nor eIF4A. In FIG. 12B, 15d-PGJ2 impairs the binding of a cap-dependent mRNA to ribosomes. Biotinylated β-globin mRNAs were incubated in RRL for 60 minutes (lanes 1-3) or for 10 minutes (lanes 4-6) in the presence of vehicle (lanes 1 and 3), 10 μM of 15d-PGJ2 (lanes 2 and 5), or 50 μM of 15d-PGJ2 (lanes 3 and 6). Ribosomal pull-down experiments were performed as described below. Western-blot analyses were performed with anti-rps6 and anti-TIAR antibodies.

### Experimental Example 4

### eIF4A as a Cytoplasmic Target of 15d-PGJ2

Subcellular localization of 15d-PGJ2 was investigated using an immunocytochemical method. Visualization of 15d-PGJ2 was accomplished by treatment with biotinylated 15d-PGJ2 followed by treatment with streptavidin-conjugated fluorescein isothiocyanate (FITC). In FIG. 6A, HeLa cells were grown on cover slips and then treated with biotinylated 15d-PGJ2 (50 μM; a, b, d, e, g, and h) or biotinylated PGE2 (50 μM; c, f, and i) for 1 hour. Immunocytochemical analyses were performed with primary antibodies against eIF3b (a, c, d, f, g, and i) and L28 antibodies (b, e, and h), and the results are shown in FIG. 6A. As shown in FIG. 6A, biotinylated chemicals were visualized with FITC-conjugated streptavidin, wherein arrows indicate SGs induced by biotinylated 15d-PGJ2.15d-PGJ2 molecules localized to both the nucleus and the cytoplasm. Interestingly, 15d-PGJ2 molecules were localized at SGs induced by 15d-PGJ2, as indicated by co-localization with the SG marker eIF3b (FIG. 6A, panels a, d, and g with yellow arrows). By contrast, biotinylated PGE2 was rather evenly distributed in the cytoplasm and SGs were not induced by PGE2 (FIG. 6A, panels c, f, and i). The large ribosomal subunit, which was visualized by the ribosomal protein L28, was not co-localized with 15d-PGJ2 (FIG. 6A, panels b, e, and h). This indicates that 15d-PGJ2-induced SGs contain high levels of 15d-PGJ2, possibly by complexing with a SG component.

The requirements of initiation factors vary in different internal ribosome entry sites (IRESes), so the target of 15d-PGJ2 was investigated by analyzing the effects of 15d-PGJ2 on the activities of various IRESes (Jang, 2006). For example, eIF4A, eIF4B and eIF4G have important roles in encephalomyocarditis virus (EMCV) IRES-dependent translation (Jang, 2006). The eIF2 ternary complex and eIF3 are needed for hepatitis C virus (HCV) IRES-dependent translation, whereas no translational initiation factor is needed for cricket paralysis virus (CrPV) IRES-dependent translation (Pisarev et al., 2005). In FIG. 6B, monocistronic mRNAs with cap-structure (1 and 2), EMCV IRES (1 and 3), HCV IRES (1 and 4), and CrPV IRES (provided by Dr. Peter Sarnow, Stanford University) (1 and 5) were translated in HeLa lysates for 1 hour in the presence (2-4) or absence (1) of 15d-PGJ2 (50 μM). Various IRES activities (RLUs of 20,000˜75,000) were observed from the mock-treated HeLa lysates. Luciferase activities in the translation mixtures containing 15d-PGJ2 were normalized to those in the corresponding translation mixtures without 15d-PGJ2, and are shown as columns (mean values). FIG. 6B reveals that Cap-dependent and EMCV IRES-dependent translation was inhibited by 15d-PGJ2 treatment (FIG. 6B, lanes 2 and 3), but HCV IRES- and CrPV IRES-dependent translation was not (FIG. 6B, lanes 4 and 5). These data indicate that eIF4G and eIF4A are potential targets of 15d-PGJ2.

Biotin pull-down experiments using biotinylated 15d-PGJ2 were performed as sown in FIG. 6C. Cytoplasmic HeLa lysates (1 mg) were treated with 50 μM of biotinylated PGE2 (2) and 50 μM biotinylated 15d-PGJ2 (3) for 1 hour at 30° C. and then streptavidin pull-down was performed as described in experimental procedures. Resin-bound proteins were analyzed by Western-blot analyses with antibodies against eIF4GI, eIF4AI, eIF4E, poly(A)-binding protein (PABP), and eIF3c, and the results are shown in FIG. 6C. Of the translation factors tested, only eIF4A was precipitated by streptavidin agarose beads from cytoplasmic HeLa cell extracts treated with biotinylated 15d-PGJ2 (FIG. 6C, panel eIF4A1).

In FIG. 6D, purified His-eIF4A1 (6 μg) was incubated with 50 μM of biotinylated PGE2 (1) or biotinylated 15d-PGJ2 (2), and then precipitated by streptavidin-sepharose. The resin-bound proteins were then analyzed by Comassie blue staining, and the results are shown in FIG. 6D. FIG. 6D reveals that other eIF4F proteins such as the scaffold protein eIF4G, cap-binding protein eIF4E, PABP, and eIF3, which bridges the eIF4G and the small ribosomal subunit, did not have a direct interaction with 15d-PGJ2. Direct interaction between eIF4A and 15d-PGJ2 was confirmed using purified eIF4 μl proteins (provided by Dr. Nadejda Korneeva, Louisiana State University). The recombinant eIF4 μl proteins were precipitated by biotin-15d-PGJ2 but not by biotin-PGE2 (FIG. 6D, lanes 1 and 2).

15d-PGJ2 contains an electrophilic carbon center susceptible to undergoing addition reactions (Michael addition) with nucleophiles such as the free sulfhydryl group of cysteine residues in cellular proteins (Straus and Glass, 2001). Human eIF4 μl contains four cysteine residues, 66C, 131C, 134C, and 264C, which are potential target sites of 15d-PGJ2. To determine which cysteine residues are involved in 15d-PGJ2-binding, the effects of cysteine to serine mutations on 15d-PGJ2-binding were monitored in FIG. 6E. 293T cells were transfected with the wild-type (WT, lane 1) or mutant (lanes 2-4) FLAG-eIF4A1s (provided by Dr. Yongjun Dang and Dr. Jian Liu, Johns Hopkins), and an immunoprecipitation was performed on the 293T cells as described above. FIG. 6E reveals that a derivative of eIF4A with the C264S mutation could not interact with 15d-PGJ2 (FIG. 6E, lane 4); however, other mutant forms of eIF4A bound to 15d-PGJ2 (lanes 2 and 3 in FIG. 6E). These data indicate that 15d-PGJ2 directly binds to the cysteine residue 264 of the eIF4A protein.

The mechanism by which 15d-PGJ2 inhibits translation was investigated by monitoring the effects of 15d-PGJ2 on translational initiation complex formation as shown FIG. 7A. eIF4G is a scaffold protein that recruits eIF4E, eIF4A, eIF3, and PABP into the translational initiation complex. The effect of 15d-PGJ2 on the eIF4GI-eIF4A interaction was monitored by a co-immunoprecipitation assay. 293T cells were co-transfected with HA-eIF4B and FLAG-eIF4A1 (provided by Dr. Yongjun Dang and Dr. Jian Liu, Johns Hopkins). Cells were lysed then treated with 50 μM of PGE2 or 15d-PGJ2 at 30° C. for 1 hour. Immunoprecipitation was performed with an anti-FLAG antibody. Western-blot analysis was performed with anti-FLAG, anti-HA, and anti-eIF4GI antibodies. eIF4GI was co-precipitated with eIF4A1 from mock-treated or PGE2-treated (FIG. 7A, lane 1) cell extracts; however, eIF4GI was not co-precipitated with eIF4A1 from the cell extract treated with 15d-PGJ2 (FIG. 7A, lane 2). eIF4B, which directly interacts with eIF4A, was co-precipitated with eIF4A1 regardless of whether the cell extracts had been treated with 15d-PGJ2 (FIG. 7A, panel HA-eIF4B in lanes 1 and 2). These data indicate that 15d-PGJ2 blocks the eIF4A-eIF4G interaction but not the eIF4A-eIF4B interaction.

The effect of 15d-PGJ2 on cap-binding protein complex formation was monitored by analyzing components in the protein complex precipitated with ⁷methyl GTP resin. Proteins in the eIF4F complex were analyzed using a ⁷methyl GTP resin and cytoplasmic HeLa lysates treated with vehicle (3), 50 μM of 15d-PGJ2 (2), or 50 μM of PGE2 (1) for 1 hour. Resin-bound proteins were washed three times and then analyzed by Western-blot assays with antibodies against eIF4GI, eIF3b, PABP, eIF4AI, actin, or eIF4E. eIF4E, eIF4GI and eIF4 μl were found in the precipitates from mock-treated and PGE2-treated HeLa cell extracts (FIG. 7B, lanes 3 and 1). eIF4E and eIF4GI were detected in the ⁷methyl GTP resin-bound protein complex even after 15d-PGJ2 treatment (FIG. 7B, lane 2). By contrast, eIF4 μl was not co-precipitated with eIF4GI after 15d-PGJ2 treatment (FIG. 7B, lane 2). These data also indicate that 15d-PGJ2 inhibits the eIF4G-eIF4A interaction.

eIF4A has RNA-binding activity (Low et al., 2005). The effect of 15d-PGJ2 on the RNA-binding activity of eIF4A was monitored using β-globin mRNA as shown in FIG. 7C. Purified His-eIF4A protein (2 μg, upper panel) and 293T cell lysate containing overexpressed FLAG-eIF4A (2 mg, lower panel) were incubated with 50 μM of PGE2 (1 and 3) or 15d-PGJ2 (2 and 4) for 1 hour. The biotinylated RNA (1 μg) β-globin mRNAs were incubated with the pretreated purified eIF4A1 or cell lysate in the presence of RNasin and nonspecific competitor tRNAs for 1 hour. RNA-bound proteins were precipitated by a streptavidin-agarose resin and then visualized by silver staining (upper panel) or Western-blot analysis with an anti-FLAG antibody (lower panel). FIG. 7C reveals that binding of purified eIF4A1 to the β-globin mRNA was increased with 15d-PGJ2 treatment (FIG. 7C, panel His-eIF4A in lanes 1 and 2). Similarly, the RNA-binding activity of FLAG-eIF4A protein expressed in mammalian cells was also increased after treatment with 15d-PGJ2, as shown by binding to β-globin mRNA (FIG. 7C, panel FLAG-eIF4A in lanes 1 and 2). The implications of this phenomenon are discussed below.

To confirm that eIF4A is the main target of 15d-PGJ2 involved in inhibition of translation, the effect of eIF4A1 supplementation on the inhibition of translation by 15d-PGJ2 was monitored as shown in FIG. 7D. In vitro translation was performed in RRL (Promega) with a Renilla luciferase mRNA (40 nM) for 1 hour with the additional purified His-eIF4 μl protein (0 ng, lanes 1 and 2; 250 ng, lanes 3 and 4; 500 ng, lanes 5 and 6). 15d-PGJ2 (50 μM) was added to the translation mixtures shown in lanes 2, 4, and 6. Luciferase activities with (2, 4, and 6) 15d-PGJ2 treatment were compared with those without (1, 3, and 5) 15d-PGJ2 treatment in the presence of additional eIF4A at particular concentrations and are shown as columns (mean values). FIG. 7D reveals that addition of purified eIF4 μl restored translation activity of the in vitro translation mixture treated with 15d-PGJ2 in a dose-dependent manner (FIG. 7D, lanes 2, 4, and 6).

Furthermore, the effects overproduction of eIF4A and its derivative with C264S mutation on the translational inhibition by 15d-PGJ2 were monitored by using HeLa cell transfection as shown in FIG. 7E. HeLa cells were grown on cover slips and transfected with a FLAG vector, plasmid FLAG-eIF4Awt expressing the wild type eIF4A tagged with FLAG, or plasmid FLAG-eIF4A^{C264S} expressing a C264S mutant eIF4A tagged with FLAG. After 48 hours of incubation, cells were treated with the chemicals at the concentrations indicated for 30 minutes. Immunocytochemical analyses were performed with eIF4GI and FLAG antibodies and the results are shown in FIG. 7E. FIG. 7E reveals that the cells overexpressing wild type eIF4A were resistant to SG formation by 15d-PGJ2 at the 50 μM (FIG. 7E, green cells on panel f) while untransfected cells form SGs at this condition (FIG. 7E, red cells on panel f). At higher concentration of 15d-PGJ2 (100 μM), however, SG formation was observed in the cells overexpressing wild type eIF4A (FIG. 7E, yellow dots in green cells on panel g). Importantly, cells overexpressing C264S mutant eIF4A, which does not bind to 15d-PGJ2, were resistant to SG formation by 15d-PGJ2 at both 50 μM and 100 μM (FIG. 7E, green cells on panels j and k). On the contrary, overexpression of eIF4A and its derivative did not inhibit SG formation by SA (FIG. 7E, green cells on panels h and l). Taken together, these data strongly indicate that 15d-PGJ2 blocks translation through direct binding to the eIF4A protein.

What is claimed is:

1. A method of screening an anti-cancer or anti-inflammatory drug comprising the steps of:
contacting a candidate compound to an animal or plant cell,
measuring inhibition of translation initiated by translational initiation factor eIF4A in vivo or in vitro, and
determining the compound as an anti-cancer or anti-inflammatory drug when the compound causes the inhibition of translation.

2. The method according to claim 1, wherein the inhibition of translation is measured by observing stress granule formation in the cell.

3. The method according to claim 1, wherein the inhibition of translation is measured by monitoring the presence of blockade of the interaction between eIF4A and eIF4G.

4. The method according to claim 1, wherein the inhibition of translation is measured by monitoring the presence of interaction between the compound and cysteine 264 of eIF4A.

5. A method of translation inhibition by treating a compound capable of blocking translational initiation factor eIF4A and eIF4G interaction.

6. A method of translation inhibition by treating a compound capable of binding to cysteine 264 of translational initiation factor eIF4A to an animal or an animal cell, to inactivate eIF4A.

7. The method according to claim 6, wherein the compound has a cyclopentenone ring.

8. The method according to claim 7, wherein the compounds are 15-deoxy-delta 12,14-prostaglandin J2 (15d-PGJ2) and prostaglandin A1 (PGA1).

9. A method of anti-inflammation or anti-cancer by administering a compound capable of binding to cysteine 264 of translational initiation factor eIF4A to a subject suffering with inflammation or cancer.

10. The method according to claim 9, wherein the compound has a cyclopentenone ring.

11. The method according to claim 10, wherein the compound is 15-deoxy-delta 12,14-prostaglandin J2 (15d-PGJ2) and prostaglandin A1 (PGA1).

12. A derivative of 15d-PGJ2 or PGA1 targeting eIF4A useful as an anti-cancer and anti-inflammation drug.

13. The derivative according to claim 12, containing cyclopentenone ring with modifications in side chains as an anti-inflammatory and anti-cancer drug.


SEQUENCE LISTING (1 sequence)

SEQ ID NO: 1 - protein, 406 aa, Artificial Sequence
  Amino acid sequence of eIF4A
MSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLI